(19) United States
(12) Patent Application Publication
(10) Pub. No.: US 2018/0231554 A1
(43) Pub. Date: Aug. 16, 2018

(54) PROGRAMMED CELL DEATH (PD-1) INHIBITOR THERAPY FOR PATIENTS WITH PD-1-EXPRESSING CANCERS

(71) Applicant: The Brigham and Women's Hospital, Inc., Boston, MA (US)

(72) Inventors: Tobias SCHATTON, Cambridge, MA (US); Sonja KLEFFEL, Boston, MA (US); Christian POSCH, Perchtoldsdorf (AT)

(73) Assignee: The Brigham and Women's Hospital, Inc., Boston, MA (US)

(21) Appl. No.: 15/751,921

(22) PCT Filed: Sep. 2, 2016
(86) PCT No.: PCT/US16/50148

§ 371 Date: Feb. 12, 2018

Related U.S. Application Data
(60) Provisional application No. 62/214,224, filed on Sep. 4, 2015.

(51) Int. Cl. G01N 33/574; C12Q 1/6886; C07K 16/28; A61P 35/00

(52) U.S. Cl. CPC G01N 33/574; C12Q 1/6886; C07K 16/2818; G01N 2333/70521; C07K 2317/76; C07K 2317/24; C07K 2317/21; A61P 35/00

(57) ABSTRACT

Described herein are biomarkers comprising mTOR, PI3K/AKT, and MAPK/ERK signaling pathway members as well as cap-dependent translation initiation factors that enable monitoring and predicting responses to PD-1 pathway blockade in patients afflicted with cancers characterized by PD-1 expression.

### RELATED APPLICATIONS

This application claims priority under 35 U.S.C. § 119(e) to U.S. Provisional Patent Application Ser. No. 62/214,224, filed Sep. 4, 2015, which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Aug. 31, 2016, is named 043214-085711-PCT_SL.txt and is 134,638 bytes in size.

### BACKGROUND OF THE INVENTION

Therapeutic antibodies targeting the programmed cell death 1 (PD-1) pathway have shown remarkable efficacy in the treatment of patients afflicted with various types of advanced-stage cancer. However, despite the success of PD-1-targeted therapies, the majority of patients still do not respond to treatment. Accordingly, a need exists for methods of discriminating responders from non-responders and of monitoring and optimizing response to anti-PD-1 therapeutic regimens. One such approach involves the detection of biomarker expression in biospecimens (e.g. tumor biopsies) obtained from patients before, during, or after treatment with PD-1 pathway inhibitors. Expression of the PD-1 ligand, PD-L1, by tumor cells and/or tumor-infiltrating lymphocytes in pre-treatment tumor biospecimens has been proposed as a possible biomarker of response to PD-1- and PD-L1-targeted agents. However, recent clinical studies did not find a significant correlation between tumor-PD-L1 status and objective response to PD-1 checkpoint blockade. These findings highlight the need for additional, more sensitive biomarkers for treatment selection and/or assessment of therapeutic response to PD-1 inhibitor therapy in patients with advanced-stage cancers.

### SUMMARY OF THE INVENTION

Cancers of various etiologies frequently contain PD-1 receptor-expressing cancer cell subpopulations. Tumor cell-expressed PD-1 modulates downstream pathways, signaling mediators of which can serve as biomarkers for predicting and monitoring response to therapeutic anti-PD-1 antibodies. It has now been discovered that mTOR, PI3K/AKT, and MAPK/ERK signaling pathway members as well as cap-dependent translation initiation factors can serve as effective biomarkers for monitoring and predicting response to PD-1 pathway blockade in patients afflicted with cancers characterized by PD-1 expression.

In one aspect, the invention provides a method of selecting a treatment correlated with a good clinical response in a human subject diagnosed with a cancer in which PD-1 is expressed, said method comprising the steps of: a) determining whether there is an increase in the level of AMP-activated protein kinase alpha and/or a decrease in the level of any one of Ribosomal protein S6, AKT, Phosphoinositide 3-kinase, Phosphoinositide-dependent protein kinase 1, Extracellular signal regulated kinase, Translation initiation factor 4E binding protein 1, Eukaryotic translation initiation factor 4B, Eukaryotic translation initiation factor 4G, mammalian target of rapamycin, PRAS40, S6 kinase 1, and src homology 2-containing protein-tyrosine-phosphatase, or any combination thereof, in a sample of the cancer, wherein said level or levels are compared to a reference level or levels; and b) selecting a treatment comprising PD-1 inhibition to administer to the human subject, thereby selecting a treatment correlated with a good clinical response in the human subject.

In one embodiment, the level is an amount of nucleic acid or protein expression.

In another embodiment, the level is an amount of phosphorylation of any one of Ribosomal protein S6, AKT, Phosphoinositide 3-kinase, Phosphoinositide-dependent protein kinase 1, Extracellular signal regulated kinase, Eukaryotic translation initiation factor 4B, Eukaryotic translation initiation factor 4G, mammalian target of rapamycin, PRAS40, S6 kinase 1, or src homology 2-containing protein-tyrosine-phosphatase, or any combination thereof.

In yet another embodiment, the cancer is melanoma, merkel cell carcinoma, lung cancer (non-small cell lung cancer and small cell lung cancer), renal cancer (e.g., renal cell carcinoma), hodkins lymphoma, glioblastoma, hepatocellular carcinoma, colorectal carcinoma (microsatellite instability-high), bladder cancer, pancreatic cancer, head and neck cancer, squamous cell carcinoma, diffuse large B-cell lymphoma, prostate cancer, ovarian cancer, endometrial carcinoma, breast cancer (e.g., triple-negative breast cancer), and any cancer subtype showing microsatellite instability.

In yet another embodiment, the treatment comprises administering a monoclonal antibody, pharmacologic agent, biologic agent, or medicinal product that inhibits PD-1 to the human subject.

In yet another embodiment, the monoclonal antibody is Pembrolizumab, Nivolumab, CT-011, AMP-244 or PDR001.

In yet another embodiment, the subject is undergoing therapy comprising PD-1 inhibition.

In yet another embodiment, the reference level is obtained from a cancer that that continues to progress following PD-1 inhibition.

In another aspect, the invention provides a method of identifying a PD-1 expressing cancer in a human subject that would fail to progress in response to PD-1 inhibition, said method comprising the steps of: a) determining whether there is an increase in the level of total and/or phosphorylated AMP-activated protein kinase alpha and/or a decrease in the level of any one of total and/or phosphorylated Ribosomal protein S6, AKT, Phosphoinositide 3-kinase, Phosphoinositide-dependent protein kinase 1, Extracellular signal regulated kinase, Translation initiation factor 4E binding protein 1, Eukaryotic translation initiation factor 4B, Eukaryotic translation initiation factor 4G, mammalian target of rapamycin, PRAS40, S6 kinase 1, and src homology 2-containing protein-tyrosine-phosphatase, or any combination thereof, in a sample of the cancer, wherein said level or levels are compared to a reference level or levels; and b) characterizing the cancer as one that would fail to progress in response to PD-1 inhibition.

In one embodiment, the level is an amount of nucleic acid or protein expression.

In another embodiment, the level is an amount of protein phosphorylation of any one of Ribosomal protein S6, AKT, Phosphoinositide 3-kinase, Phosphoinositide-dependent protein kinase 1, Extracellular signal regulated kinase, Eukaryotic translation initiation factor 4B, Eukaryotic translation initiation factor 4G, mammalian target of rapamycin, PRAS40, S6 kinase 1, or src homology 2-containing protein-tyrosine-phosphatase, or any combination thereof.

In yet another embodiment, the cancer is melanoma, merkel cell carcinoma, lung cancer (non-small cell lung cancer and small cell lung cancer), renal cancer (e.g., renal cell carcinoma), bodkins lymphoma, glioblastoma, hepatocellular carcinoma, colorectal carcinoma (microsatellite instability-high), bladder cancer, pancreatic cancer, head and neck cancer, squamous cell carcinoma, diffuse large B-cell lymphoma, prostate cancer, ovarian cancer, endometrial carcinoma, breast cancer (e.g., triple-negative breast cancer), and any cancer subtype showing microsatellite instability.

In yet another embodiment, a cancer that fails to progress is decreased in size or severity following PD-1 inhibition.

In yet another embodiment, the subject is undergoing therapy comprising PD-1 inhibition.

In yet another embodiment, the reference level is obtained from a cancer that that continues to progress following PD-1 inhibition.

In yet another aspect, the invention provides a method of treating a human subject diagnosed with a cancer in which PD-1 is expressed, said method comprising the steps of: a) administering a treatment comprising an inhibitor of PD-1 to the human subject; b) determining whether there is an increase in the level of AMP-activated protein kinase alpha and/or a decrease in the level of any one of Ribosomal protein S6, AKT, Phosphoinositide 3-kinase, Phosphoinositide-dependent protein kinase 1, Extracellular signal regulated kinase, Translation initiation factor 4E binding protein 1, Eukaryotic translation initiation factor 4B, Eukaryotic translation initiation factor 4G, mammalian target of rapamycin, PRAS40, S6 kinase 1, and src homology 2-containing protein-tyrosine-phosphatase, or any combination thereof, in a sample of the cancer, wherein said level or levels are compared to a reference level or levels; and c) continuing to administer a treatment comprising an inhibitor of PD-1 to the human subject, thereby treating the human subject.

In yet another aspect, the invention provides a method of selecting a treatment correlated with a good clinical response in a human subject diagnosed with a cancer in which PD-1 is expressed, said method comprising the steps of: determining whether there is at least about 5% (e.g., about 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99% or 100%) expression of AMP-activated protein kinase alpha and/or a decrease in the level of any one of Ribosomal protein S6, AKT, Phosphoinositide 3-kinase, Phosphoinositide-dependent protein kinase 1, Extracellular signal regulated kinase, Translation initiation factor 4E binding protein 1, Eukaryotic translation initiation factor 4B, Eukaryotic translation initiation factor 4G, mammalian target of rapamycin, PRAS40, S6 kinase 1, src homology 2-containing protein-tyrosine-phosphatase, or any combination thereof, in a sample of the cancer; and selecting a treatment comprising PD-1 inhibition to administer to the human subject, thereby selecting a treatment correlated with a good clinical response in the human subject.

In all aspects of the invention, the cancer in which PD-1 is expressed can comprise subsets of cells that express PD-1 and other cells that do not express PD-1 or detectable levels of PD-1.

Other features and advantages of the invention will be apparent from the Detailed Description, and from the claims. Thus, other aspects of the invention are described in the following disclosure and are within the ambit of the invention.

### BRIEF DESCRIPTION OF THE DRAWINGS

The following Detailed Description, given by way of example, but not intended to limit the invention to specific embodiments described, may be understood in conjunction with the accompanying figures, incorporated herein by reference.

FIG. 1A shows immunoblot results revealing decreased expression of phosphorylated ribosomal protein S6 (p-S6) in human PD-1 knockdown (PDCD1 shRNA-1 and shRNA-2) vs. control G3361 melanoma cells and increased expression of p-S6 and p-AKT in PD-1 overexpressing (PDCD1 OE) compared to control G3361 melanoma cells. FIG. 1B shows that melanoma-specific PD-1 knockdown (Pdcdl shRNA-1 and shRNA-2) similarly decreases and PD-1 overexpression (Pdcd1 OE) increases p-S6 expression in murine B16-F10 melanoma cells, as determined by immunoblotting.

FIG. 2 shows representative immunohistochemistry images revealing increased p-S6 (top) and p-AKT expression (bottom) in PD-1-overexpressing (enforced melanoma-PD-1 expression) compared to control C8161 human melanoma xenografts grown in highly immunocompromised (NOD/SCID IL2Rγ (−/−) knockout) mice.

FIG. 3A shows that the introduction of tyrosine to phenylalanine mutations (Y223F, Y248F, or both) to melanoma-PD-1 signaling motifs via site-directed mutagenesis results in reduced S6 phosphorylation compared to wildtype PD-1-overexpressing (PDCD1 OE) or control human C8161 melanoma cells, as determined by immunoblot analysis. FIG. 3B shows that mutagenesis of either one (Y225F, Y248F) or both melanoma-PD-1 signaling motifs in murine B16-F10 melanoma cells similarly decreases p-S6 expression compared to wildtype Pdcd1-OE or vector control lines.

FIG. 4A shows by immunoblot analysis that the engagement of PD-1 expressed by human G3361 melanoma cells by its ligand, PD-L1 (added to G3361 cultures in form of a recombinant PD-L1 Fc-fusion protein, PD-L1 Ig), promotes phosphorylation of various members of the mTOR and PI3K/AKT signaling pathway, including ribosomal protein S6, AKT (at both phosphorylation sites, serines 478 and 308), PI3K, and PDK1. FIG. 4B shows that PD-L1 Ig treatment similarly increases p-S6 and, to a lesser extent p-AKT (478) expression in B16-F10 melanoma cells compared to control Ig treatment. Additionally, PD-L1 Ig-treated B16-F10 cells demonstrate increased expression of p-ERK and decreased expression of p-AMPKa.

FIG. 5A shows by immunoblot analysis that antibody-mediated blockade of PD-1 on human G3361 melanoma cells inhibits the PD-L1 Ig-induced phosphorylation of S6, AKT (serines 473 and 308), and PI3K. FIG. 5B shows that antibody-mediated PD-1 blockade on B16-F10 melanoma cells inhibits phosphorylation of S6 compared to isotype control antibody treatment.

FIG. 6 illustrates representative immunohistochemical stainings of total SHP2 and p-SHP2 expression in pretreatment tumor biopsies obtained from melanoma patients undergoing PD-1 inhibitor treatment.

FIG. 7 shows Kaplan-Meier analyses of overall survival in melanoma patients undergoing PD-1 inhibitor treatment whose pre-treatment tumor biopsies showed high (>25%) vs. low (<25%) expression of total SHP2 (panel A) or p-SHP2 (panel B), as determined by immunohistochemical analysis as in FIG. 6. Melanoma patients with p-SHP2 high tumors showed prolonged overall survival in response to PD-1 inhibitor treatment compared to patients with p-SHP2 low tumor specimens.

FIGS. 8A-B illustrate representative immunohistochemical stainings of p-S6 and total S6 (t-S6) expression in pretreatment tumor biopsies obtained from melanoma patients undergoing PD-1 inhibitor treatment that showed high (>25% p-S6 expression by melanoma cells, FIG. 8A) vs. low p-S6 expression (<25%) by melanoma cells (FIG. 8B).

FIG. 9 shows expression of p-S6 ribosomal protein by melanoma cells in tumor biospecimens obtained from n=11 patients with stage IV melanoma before treatment start compared to that in patient-matched progressive lesions sampled after initation of anti-PD-1 antibody therapy. Melanoma-p-S6 expression was determined by immunohistochemical analysis and graded by three independent investigators blinded to the study outcome on a scale of 0-4 (0: no p-S6 expression by melanoma cells; 1: p-S6 expression in 1%-25%; 2: 26%-50%; 3:51%-75%; 4: >75% of melanoma cells).

FIGS. 10A-10B depict a Kaplan-Meier analysis of progression-free survival (FIG. 10A) and overall survival (FIG. 10B) in n=34 melanoma patients undergoing PD-1 inhibitor treatment whose pre-treatment tumor biopsies showed high (>25%, n=20) vs. low (<25%, n=14) expression of p-S6, as determined by immunohistochemical analysis as in FIG. 8. Melanoma patients with p-S6 high tumors showed significantly prolonged progression-free and overall survival in response to PD-1 inhibitor treatment compared to patients with p-S6 low tumor specimens.

FIG. 11 depicts representative immunohistochemical stainings of p-AKT, p-PI3K, and p-PDK1 expression in pretreatment tumor biopsies obtained from melanoma patients undergoing PD-1 inhibitor treatment.

FIG. 12 shows Kaplan-Meier analyses of progression-free survival in melanoma patients undergoing PD-1 inhibitor treatment whose pre-treatment tumor biopsies showed high (>25%) vs. low (<25%) expression of p-AKT (left panel), p-PI3K (center panel), or p-PDK1 (right panel), as determined by immunohistochemical analysis as in FIG. 11. Melanoma patients with tumors high in p-AKT, p-PI3K, or p-PDK1 showed prolonged progression-free survival in response to PD-1 inhibitor treatment compared to patients with tumor specimens low in the respective markers.

FIG. 13A shows that PD-L1 Ig treatment of human G3361 melanoma cells increases expression of eukaryotic translation initiation factor 4E binding protein 1 (4E-BP1) compared to control Ig treatment and that antibody-mediated PD-1 blockade can inhibit the PD-L1 Ig-induced expression of 4E-BP1, as determined by immunoblotting. FIG. 13B shows that PD-L1 Ig treatment also induces expression of the translation initiation factor, eIF-4G, in human G3361 melanoma cells.

FIGS. 14A-14B illustrate representative immunohistochemical stainings of 4E-PB1 expression in pretreatment tumor biopsies obtained from melanoma patients undergoing PD-1 inhibitor treatment that showed high (>25% 4E-BP1 expression by melanoma cells, FIG. 14A) vs. low 4E-BP1 expression (<25%) by melanoma cells (FIG. 14B). Depicted is IHC staining of pre-treatment tumor biopsies of melanoma patients undergoing PD-1 inhibitor trials and shows high expression of 4E-BP1 (greater than 25%) in the majority of patients with good clinical response (long progression-free and overall survival, FIG. 14A) and low 4E-BP1 expression (less than 25%) in patients with poor clinical response to PD-1 antibody treatment (short progression-free and overall survival, FIG. 14B).

FIG. 15 shows a Kaplan-Meier analysis of progression-free survival in melanoma patients undergoing PD-1 inhibitor treatment whose pre-treatment tumor biopsies showed high (>25%) vs. low (<25%) expression of 4E-BP1, as determined by immunohistochemical analysis as in FIG. 14. Melanoma patients with 4E-BP1 high tumors showed prolonged progression-free survival in response to PD-1 inhibitor treatment compared to patients with 4E-BP1 low tumor specimens.

FIG. 16A depicts a representative immunohistochemical staining of p-eIF-4G expression in a pretreatment tumor biopsy obtained from a melanoma patient undergoing PD-1 inhibitor treatment. FIGS. 16B and 16C show Kaplan-Meier analyses of progression-free and overall survival, respectively, in melanoma patients undergoing PD-1 inhibitor treatment whose pre-treatment tumor biopsies showed high (>50%) vs. low (<50%) expression of p-eIF-4G, as determined by immunohistochemical analysis as in panel A. Melanoma patients with p-eIF-4G high tumors showed significantly prolonged progression-free and overall survival in response to PD-1 inhibitor treatment compared to patients with p-eIF-4G low tumor specimens.

FIG. 17A shows that treatment of PD-1-expressing Merkel cell carcinoma (MCC) cell lines, MKL1, MKL2, MS-1, and WaGa, with recombinant PD-L1 Ig and PD-L2 Ig promotes phosphorylation of mTOR and PRAS40 compared to control Ig treatment, as determined by immunoblot analysis. FIG. 17B shows pixel densities for immunoblot bands as in FIG. 17A (*, P<0.05).

FIG. 18A shows by immunoblot analysis that PD-L1 Ig and PD-L2 Ig treatment of MKL2 MCC cells induces phosphorylation of mTOR, PRAS40, S6K1, and eIF-4B compared to control Ig treatment and that the enhanced phosphorylation of these molecules can be inhibited using a PD-1 blocking antibody. FIG. 18B shows relative pixel densities and percent reduction of pixel density for PD-L1 Ig (top) and PD-L2 Ig (bottom)-treated MKL2 cells co-incubated with PD-1 blocking antibody vs. isotype control antibody for immunoblot bands as in FIG. 18A.

FIG. 19A shows decreased expression of p-mTOR in MCC tumor xenografts grown in highly immunocompromised (NOD/SCID IL2Rγ (−/−) knockout) mice treated with a PD-1 blocking antibody (three right bands) compared to isotype control antibody (three left bands). FIG. 19B shows pixel densities of immunoblot bands as in FIG. 19A (*, P<0.05).

### DETAILED DESCRIPTION OF THE INVENTION

### Definitions

Unless otherwise defined, all technical and scientific terms used herein have the same meaning as commonly understood by one of ordinary skill in the art to which this invention belongs. In case of conflict, the present application, including defmitions will control.

A “subject” is a vertebrate, including any member of the class mammalia, including humans, domestic and farm animals, and zoo, sports or pet animals, such as mouse, rabbit, pig, sheep, goat, cattle and higher primates.

As used herein, the terms “treat,” “treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, “proliferative growth disorder, “neoplastic disease,” “tumor”, “cancer” are used interchangeably as used herein refers to a condition characterized by uncontrolled, abnormal growth of cells.

As used herein, a “good clinical response” to PD-1 antibody treatment or other treatment comprising inhibition of PD-1 refers to a duration of time that is at least about 50 days or more, during which a patient demonstrates progression-free survival.

As used herein, a “poor clinical response” to PD-1 antibody treatment or other treatment comprising inhibition of PD-1 refers to a duration of time that is about 30 days or less, during which patient demonstrates progression-free survival.

As used herein, the term “progression-free survival” refers to the time from the first administration of anti-PD-1-based therapy to the first documented radiographic evidence of progressive disease. The term “overall survival” is defined as the time from the first administration of anti-PD-1 therapy to the date of death, regardless of cause.

As used herein, a cancer that “fails to progress” decreases in size or severity following PD-1 inhibition after about 300 days or less (e.g., after about 50 days).

As used herein, a PD-1 inhibitor is any pharmacologic or biologic agent or medicinal product that reduces the activity or expression of PD-1 and/or modulates PD-1 interactions with its ligands and/or other molecules and/or inhibits PD-1 signaling and/or pathway activity.

Unless specifically stated or clear from context, as used herein, the term “about” is understood as within a range of normal tolerance in the art, for example within 2 standard deviations of the mean. “About” is understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used herein “a decrease in expression or phosphorylation” refers to an amount of gene expression, protein expression or protein phosphorylation that is at least about 0.05 fold less (for example 0.1, 0.2, 0.3, 0.4, 0.5, 1, 5, 10, 25, 50, 100, 1000, 10,000-fold or more less) than the amount of gene expression, protein expression or protein phosphorylation in a subject not undergoing PD-1 inhibition or in a subject prior to undergoing PD-1 inhibition according to the methods described herein. “Decreased” as it refers to gene expression, protein expression or protein phosphorylation also means at least about 5% less (for example 5, 6, 7, 8, 9, 10, 15, 20, 25, 30, 35, 40, 45, 50, 55, 60, 65, 70, 75, 80, 85, 90, 95, 99 or 100%) than the amount of gene expression, protein expression or protein phosphorylation in a subject not undergoing PD-1 inhibition or in a subject prior to undergoing PD-1 inhibition according to the methods described herein. Amounts can be measured according to methods known in the art for determining amounts of gene expression, protein expression or protein phosphorylation.

As used herein “an increase in expression or phosphorylation” refers to an amount of gene expression, protein expression or protein phosphorylation that is at least about 0.05 fold more (for example 0.1, 0.2, 0.3, 0.4, 0.5, 1, 5, 10, 25, 50, 100, 1000, 10,000-fold or more) than the amount of gene expression, protein expression or protein phosphorylation in a subject not undergoing PD-1 inhibition or in a subject prior to undergoing PD-1 inhibition according to the methods described herein. “Increased” as it refers to gene expression, protein expression or protein phosphorylation also means at least about 5% more (for example 5, 6, 7, 8, 9, 10, 15, 20, 25, 30, 35, 40, 45, 50, 55, 60, 65, 70, 75, 80, 85, 90, 95, 99 or 100%) than the amount of gene expression, protein expression or protein phosphorylation in a subject not undergoing PD-1 inhibition or in a subject prior to undergoing PD-1 inhibition according to the methods described herein. Amounts can be measured according to methods known in the art for determining amounts of gene expression, protein expression or protein phosphorylation.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

As used herein, the term “reference level” refers to the level of expression of a biomarker in a known sample against which another test sample is compared. A reference level can be obtained, for example, from a known cancer sample from a different individual (e.g., not the individual being tested) that continues to progress following PD-1 inhibition. The reference level may be determined before and/or after PD-1 inhibition and optionally, from samples obtained from the same subject before and/or after PD-1 inhibition. A known sample can also be obtained by pooling samples from a plurality of individuals to produce a reference level over an averaged population. A “level” can be an amount of nucleic acid expression, protein expression, or phosphorylation of a biomarker.

As used herein, “PD-1 expression by cancer cells” refers to immunofluorescence, immunohistochemistry, flow cytometry, immunoblot, and/or in situ hybridization-based detection of PD-1 by one or more hematopoietic lineage-negative (e.g. CD45-negative), and/or endothelial marker-negative (e.g. CD31-negative), and/or tumor-associated antigen-positive (e.g. MART-1 (melanoma), cytokeratin-20 (Merkel cell carcinoma), EpCAM/ESA (lung and breast cancer), pancytokeratin/vimentin/PAX2/PAX8 (renal cell carcinoma), cytokeratin-20 (bladder cancer)), and/or morphologically distinguished cancer cells within tumor sections. Cancers known to be characterized by PD-1 expression include, but are not limited to, melanoma, merkel cell carcinoma, lung cancer (non-small cell lung cancer and small cell lung cancer), renal cancer (e.g., renal cell carcinoma), bodkins lymphoma, glioblastoma, hepatocellular carcinoma, colorectal carcinoma (microsatellite instability-high), bladder cancer, pancreatic cancer, head and neck cancer, squamous cell carcinoma, diffuse large B-cell lymphoma, prostate cancer, ovarian cancer, endometrial carcinoma, breast cancer (e.g., triple-negative breast cancer), and any cancer subtype showing microsatellite instability. PD-1 expression, signaling, or activity can also be indicated by p-S6 expression.

In this disclosure, “comprises,” “comprising,” “containing” and “having” and the like can have the meaning ascribed to them in U.S. Patent law and can mean “includes,” “including,” and the like; “consisting essentially” of or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Other definitions appear in context throughout this disclosure.

### Compositions and Methods of the Invention

Described herein are mTOR, PI3K/AKT/, and MAPK/ERK signaling pathway members as well as cap-dependent translation initiation factors that can serve as effective biomarkers for monitoring and predicting response to PD-1 pathway blockade in patients afflicted with cancers containing PD-1-expressing cancer cell subsets.

Ribosomal protein S6 (p-S6) is a component of the 40S ribosomal subunit and is involved in protein translation. The human nucleotide and amino acid sequences encoding Ribosomal protein S6 are known in the art and can be located, for example, at GenBank accession numbers BC094826.1 (SEQ ID NO: 1) and AAA60289.1 (SEQ ID NO: 2), respectively. Antibodies for use in monitoring Ribosomal protein S6 levels are also well known in the art and are described, for example, by Fonseca, B. D. et al. (2011) J Biol Chem 286, 27111-22, Guertin et al. (2009) Cancer Cell. 15:148-59, Engelman et al. (2008) Nat Med. 14:1351-6, Lan et al. (2012) Am J Physiol Renal Physiol. 302:F1210-23, Posch et al. (2013) Proc Natl Acad Sci. 110:4015-20, Mueller et al. (2012) Neuro Oncol. 14:1146-52, Annovazzi et al. (2009) Anticancer Res. 29:3087-94 and Rojo et al. (2007) Clin Cancer Res. 13:81-9. Levels of Ribosomal protein S6 are decreased in response to PD-1 inhibition in cancers containing PD-1-expressing cancer cell subsets.

AKT is a serine/threonine-specific protein kinase that functions in cellular survival and metabolism by regulating many downstream effectors. The human nucleotide and amino acid sequences encoding AKT are known in the art and can be located, for example, at GenBank accession numbers M63167.1 (SEQ ID NO: 3) and AAA36539.1 (SEQ ID NO: 4), for AKT 1, respectively. Sequences encoding AKT 2 and 3 are also are known in the art. Antibodies for use in monitoring AKT levels (e.g., AKT 1, 2 and 3) are also well known in the art and are described, for example, by Michels, S. et al. (2013) Cancer Res 73, 2518-28, Guertin et al. (2009) Cancer Cell. 15:148-59, Engelman et al. (2008) Nat Med. 14:1351-6, Kippenberger et al. (2010) Biochim Biophys Acta. 1803:940-50, Posch et al. (2013) Proc Natl Acad Sci. 110:4015-20, Annovazzi et al. (2009) Anticancer Res. 29:3087-94 and Rojo et al. (2007) Clin Cancer Res. 13:81-9. Levels of AKT are decreased in response to PD-1 inhibition in cancers containing PD-1-expressing cancer cell subsets.

Phosphoinositide 3-kinase or PI3K catalyzes the production of phosphatidylinositol-3,4,5-triphosphate by phosphorylating phosphatidylinositol (PI), phosphatidylinositol-4-phosphate (PIP), and phosphatidylinositol-4,5-bisphosphate (PIP2). The human nucleotide and amino acid sequences encoding PI3K (e.g., regulatory subunit 1 and 2 (beta)) are known in the art and can be located, for example, at GenBank accession numbers, P27986.2 (SEQ ID NO: 5), BC011917.2 (SEQ ID NO: 6) and O00459.2 (SEQ ID NO: 7), BC030815.1 (SEQ ID NO: 8) respectively. Antibodies for use in monitoring PI3K levels are also well known in the art and are described, for example, by Rush, J. et al. (2005) Nat Biotechnol 23, 94-101, Jian et al. (2015) Exp Eye Res. 132:34-47, Li et al. (2012) Ann Surg Oncol. 19:145-53 and Gu et al. (2009) Cancer Res. 69:9465-72. Levels of Phosphoinositide 3-kinase are decreased in response to PD-1 inhibition in cancers containing PD-1-expressing cancer cell subsets.

Phosphoinositide-dependent protein kinase 1 or PDK1 is involved in the regulation of a wide variety of processes, including cell proliferation, differentiation and apoptosis. The human nucleotide and amino acid sequences encoding PDK1 are known in the art and can be located, for example, at GenBank accession numbers AF017995.1 (SEQ ID NO: 9) and O15530.1 (SEQ ID NO: 10) respectively. Antibodies for use in monitoring PDK1 levels are also well known in the art and are described, for example, by Sawitzky, M. et al. (2012) PLoS One 7, e39711, Arsenic (2014) Diagn Pathol. 9:82, and Rodriguez et al. (2009) Am J Pathol. 174:2051-60. Levels of Phosphoinositide-dependent protein kinase 1 are decreased in response to PD-1 inhibition in cancers containing PD-1-expressing cancer cell subsets.

Extracellular signal regulated kinase or ERK is a protein kinase intracellular signaling molecule involved in functions including the regulation of meiosis, mitosis, and postmitotic functions in differentiated cells. Many different stimuli, including growth factors, cytokines, virus infection, ligands for heterotrimeric G protein-coupled receptors, transforming agents, and carcinogens, activate the ERK pathway. The human nucleotide and amino acid sequences encoding ERK are known in the art and can be located, for example, at GenBank accession numbers BC099905.1 (SEQ ID NO: 11) and NP_620407.1 (SEQ ID NO: 12), respectively. Antibodies for use in monitoring ERK levels are also well known in the art and are described, for example, by Michels, S. et al. (2013) Cancer Res 73, 2518-28, Engelman et al. (2008) Nat Med. 14:1351-6, Kippenberger et al. (2010) Biochim Biophys Acta. 1803:940-50, Faber et al. (2011) Cancer Discov. 1:352-65, Syme et al. (2004) J Biol Chem. 280:11281-8, Posch et al. (2013) Proc Natl Acad Sci. 110:4015-20, and Rojo et al. (2007) Clin Cancer Res. 13:81-9. Levels of extracellular signal regulated kinase are decreased in response to PD-1 inhibition in cancers containing PD-1-expressing cancer cell subsets.

AMP-activated protein kinase or AMPK alpha is a protein kinase that regulates the metabolism of fatty acids and glycogen, protein synthesis, cell growth, and blood flow. The human nucleotide and amino acid sequences encoding AMPK alpha are known in the art and can be located, for example, at GenBank accession numbers AB022017.1 (SEQ ID NO: 13) and Q13131.4 (SEQ ID NO: 14), respectively. Antibodies for use in monitoring p-AMPK alpha levels (e.g., alpha 1 and 2 isoforms) are also well known in the art and are described, for example, by Zhang, J. et al. (2013) Nat Cell Biol., Escobar et al. (2015) J Surg Res. 194:164-72, Fleming et al. (2014) Histopathology 64:477-83 and Fullerton et al. (2013) Nat Med. 19:1649-54. Levels of AMPK alpha are increased in response to PD-1 inhibition in cancers containing PD-1-expressing cancer cell subsets.

Eukaryotic translation initiation factor 4B or eIF-4B assists the eIF4F complex in translation initiation. The human nucleotide and amino acid sequences encoding eIF-4B are known in the art and can be located, for example, at GenBank accession number BC073139.1 (SEQ ID NOS 15-16, respectively). Antibodies for use in monitoring eIF-4B levels are also well known in the art and are described, for example, by Peiretti et al. (2004) EMBO J. 23, 1761-69, Choi et al. (2015) Hum Pathol. 46:753-60, Degen et al. (2013) PLoS ONE. 8:e78979. Levels of Eukaryotic translation initiation factor 4B are decreased in response to PD-1 inhibition in cancers containing PD-1-expressing cancer cell subsets.

Eukaryotic translation initiation factor 4G or eIF-4G is involved in bringing mRNA to the ribosome for translation initiation. The human nucleotide and amino acid sequences encoding eIF-4G are known in the art and can be located, for example, at GenBank accession number NC_000003.12. Antibodies for use in monitoring eIF-4G levels are also well known in the art and are described, for example, by Mueller et al. (2011) PLoS One. 6:e23780, Tu et al. (2010) Mol Cancer. 16:9-78, and El-Salem et al. (2007) Lab Invest. 87:29-39. Levels of Eukaryotic translation initiation factor 4G are decreased in response to PD-1 inhibition in cancers containing PD-1-expressing cancer cell subsets.

Translation initiation factor 4E binding protein 1 or (4E-BP1) inhibits cap-dependent translation by binding to the translation initiation factor eIF-4E. The human nucleotide and amino acid sequences encoding 4E-BPlare known in the art and can be located, for example, at GenBank accession numbers NM_004095.3 (SEQ ID NO: 17) and NP_004086.1 (SEQ ID NO: 18), respectively. Antibodies for use in monitoring 4E-BP1 levels are also well known in the art and are described, for example, by Naito, T. et al. (2013) J Biol Chem 288, 21074-81, Engelman et al. (2008) Nat Med. 14:1351-6, Mueller et al. (2012) Neuro Oncol. 14:1146-52, Ma et al. (2015) Mol Med Rep., De Martino et al. (2014) Nat Med. 21:601-13, Rojo et al. (2007) Clin Cancer Res. 13:81-9, and El-Salem et al. (2007) Lab Invest. 87:29-39. Levels of Translation initiation factor 4E binding protein 1 are decreased in response to PD-1 inhibition in cancers containing PD-1-expressing cancer cell subsets.

The mammalian target of rapamycin or mTOR is a Ser/Thr protein kinase that functions as an ATP and amino acid sensor to balance nutrient availability and cell growth. The human nucleotide and amino acid sequences encoding mTOR are known in the art and can be located, for example, at GenBank accession number NM_004958.3 (SEQ ID NOS 19-20, respectively). Antibodies for use in monitoring mTOR levels are also well known in the art and are described, for example, by Shavlakadze, T. et al. (2010) J Cell Sci, Annovazzi et al. (2009) Anticancer Res. 29:3087-94, Melling et al. (2015) Int J Clin Exp Pathol. 8:7009-15 and Fiorini et al. (2014) Am J Cancer Res. 4:907-15. Levels of mammalian target of rapamycin are decreased in response to PD-1 inhibition in cancers containing PD-1-expressing cancer cell subsets.

PRAS40 is a substrate for AKT. The human nucleotide and amino acid sequences encoding PRAS40 are known in the art and can be located, for example, at GenBank accession numbers BC007416.2 (SEQ ID NO: 21) and Q96B36.1 (SEQ ID NO: 22), respectively. Antibodies for use in monitoring PRAS40 levels are also well known in the art and are described, for example, by Roca, H. et al. (2009) Neoplasia 11, 1309-17, Faber et al. (2011) Cancer Discov. 1:352-65, Yuan et al. (2015) Oncol Lett. 9:785-89, Mueller et al. (2012) Neuro Oncol. 14:1146-52 and Holzer et al. (2011) Anticancer Res. 31:2073-81. Levels of PRAS40 are decreased in response to PD-1 inhibition in cancers containing PD-1-expressing cancer cell subsets.

S6K1 or p70 S6 kinase 1 is a mitogen activated Ser/Thr protein kinase that is required for cell growth and G1 cell cycle progression. The human nucleotide and amino acid sequences encoding S6K1 are known in the art and can be located, for example, at GenBank accession numbers AK293247.1 (SEQ ID NO: 23) and P23443.2 (SEQ ID NO: 24), respectively. Antibodies for use in monitoring S6K1 levels are also well known in the art and are described, for example, by Izumi, N. et al. (2012) Cancer Sci 103, 50-7, Ma et al. (2015) Mol Med Rep., De Martino et al. (2014) Nat Med. 21:601-13, and Rojo et al. (2007) Clin Cancer Res. 13:81-9. Levels of S6 kinase 1 are decreased in response to PD-1 inhibition in cancers containing PD-1-expressing cancer cell subsets.

Src homology 2-containing protein-tyrosine-phosphatase or SHP2 (also known as PTPN11) PTPN11 is a member of the protein tyrosine phosphatase (PTP) family. There are two gene variants of SHP2. The human nucleotide sequences encoding SHP2 are known in the art and can be located, for example, at GenBank accession numbers NM_002834.3 (SEQ ID NO: 25) and NM_080601.1 (SEQ ID NO: 26). There are two protein variants of SHP2. The human amino acid sequences encoding SHP2 are known in the art and can be located, for example, at GenBank accession numbers NP_002825.3 (SEQ ID NO: 27) and NP_542168.1 (SEQ ID NO: 28). Antibodies for use in monitoring SHP2 levels are also well known in the art and are described, for example, by Leibowitz M. et al. Clin Cancer Res. 2013; 19 (4):798-808 and Han et al. J Hepatol. 2016; 63 (3):651-660. Levels of p-SHP2 are decreased in response to PD-1 inhibition in cancers containing PD-1-expressing cancer cell subsets.

Methods of the invention can be used to predict whether a cancer in a human subject will respond to a therapy that involves PD-1 inhibition. They can also be used to determine whether a cancer in a human subject is responding to a therapy involving PD-1 inhibition. A sample obtained from the cancer in question can be evaluated to determine whether there is an increase in the level of total or phosphorylated AMP-activated protein kinase alpha and/or a decrease in the level of any one of total or phosphorylated Ribosomal protein S6, AKT, Phosphoinositide 3-kinase, Phosphoinositide-dependent protein kinase 1, Extracellular signal regulated kinase, Translation initiation factor 4E binding protein 1, Eukaryotic translation initiation factor 4B, mammalian target of rapamycin, PRAS40, S6 kinase 1, and SHP2 or any combination thereof, when compared to a reference level or levels, either before or during treatment with a PD1-1 inhibitor. A sample obtained from the cancer in question can be evaluated to determine whether there is at least about 5% (e.g., about 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99% or 100%) expression of AMP-activated protein kinase alpha and/or a decrease in the level of any one of Ribosomal protein S6, AKT, Phosphoinositide 3-kinase, Phosphoinositide-dependent protein kinase 1, Extracellular signal regulated kinase, Translation initiation factor 4E binding protein 1, Eukaryotic translation initiation factor 4B, Eukaryotic translation initiation factor 4G, mammalian target of rapamycin, PRAS40, S6 kinase 1, src homology 2-containing protein-tyrosine-phosphatase, or any combination thereof, in the sample of the cancer prior to initiating PD-1 inhibition therapy. The term “any combination thereof” means that any combination of the biomarkers can be evaluated together, in addition to evaluating any single biomarker alone.

Determining the level of one or more biomarkers can involve measuring an amount of mRNA or protein expression or protein phosphorylation using methods known in the art.

1. To measure protein expression or phosphorylation in general, a western blotting technique can be employed. In this regard, a predetermined amount of protein obtained from a sample of the cancer is loaded on SDS PAGE gel, transferred onto a membrane, and reacted with an antibody with known antigenic specificity to a biomarker, and then, exposed using enhanced chemiluminescence, infrared-based, or alternative methodologies for the detection of a band. Band intensities can be determined by densitometry using commercially or publically available software well known in the art. Protein phosphorylation of any one of Ribosomal protein S6, AKT, Phosphoinositide 3-kinase, Phosphoinositide-dependent protein kinase 1, Extracellular signal regulated kinase, Eukaryotic translation initiation factor 4B, Eukaryotic translation initiation factor 4G, mammalian target of rapamycin, PRAS40, S6 kinase 1, and src homology 2-containing protein-tyrosine-phosphatase, or any combination thereof, can be measured according to methods known in the art.

In specific embodiments, an increased or decreased level of immunostaining in a sample is a level of immunostaining that would be considered higher or lower, respectively, than the level of immunostaining compared to a reference (e.g., control) by a person of ordinary skill in the art. Methods of immunostaining (also referred to as immunohistochemistry or IHC) are well known in the art.

Immunohistochemical (IHC) staining techniques are used for the visualization of antigens (e.g., p-AKT) in tissue sections. These techniques are based on the immunoreactivity and specificity of antibodies, and the chemical properties of enzymes or enzyme complexes which react with colorless substrate-chromogens to produce a colored end product. IHC staining techniques include direct and indirect methods, either of which can be used. In the direct method, the chromogen is conjugated directly to an antibody with known antigenic specificity (primary antibody). This technique allows the visualization of tissue antigens using standard light microscopy. Commercial antibodies with known antigenic specificity to each of the biomarkers of the invention are available from Cell Signaling Technologies as well as other sources.

The indirect method is a two-step method in which enzyme-labeled secondary antibodies react with the antigen-bound primary antibody. Enzyme pairs which can be used in the indirect method include peroxidase-antiperoxidase (PAP) and avidin-biotin. When the indirect method employs an avidin-biotin complex (ABC), a biotinylated secondary antibody forms a complex with peroxidase-conjugated streptavidin molecules. Specimens are incubated with a primary antibody, followed by sequential incubations with the biotinylated secondary link antibody and peroxidase labeled streptavidin. The primary antibody-secondary antibody-avidin enzyme complex is then visualized utilizing a substrate-chromogen that produces a brown pigment at the antigen site that is visible by light microscopy.

Determining the amount of biomarker present in a test sample using IHC is done in comparison to a control sample (e.g., providing a “reference level”), using either manual scoring or automated detection systems. A spectrophotometric plate reader may be used for colorimetric detection. Several types of reporters can sensitivity in an immunoassay. For example, chemiluminescent substrates have been developed which further amplify the signal and can be read on a luminescent plate reader.

In other specific embodiments, biomarkers are detected using antibody-coated microbeads, such as magnetic beads. Alternatively, the beads are internally color-coded with fluorescent dyes and the surface of the bead is tagged with an anti-biomarker marker antibody (e.g., an anti-AKT antibody) that can bind a biomarker in a test sample. The biomarker, in turn, is either directly labeled with a fluorescent tag or indirectly labeled with an anti-marker antibody conjugated to a fluorescent tag. Hence, there are two sources of color, one from the bead and the other from the fluorescent tag. Alternatively, the beads can be internally coded by different sizes.

By using a blend of different fluorescent intensities from the two dyes, as well as beads of different sizes, the assay can measure up to hundreds of different cancer markers. During the assay, a mixture containing the color/size-coded beads, fluorescence labeled anti-marker antibodies, and the sample are combined and injected into an instrument that uses precision fluidics to align the beads. The beads then pass through a laser and, on the basis of their color or size, either get sorted or measured for color intensity, which is processed into quantitative data for each reaction.

When samples are directly labeled with fluorophores, the system can read and quantitate only fluorescence on beads without removing unbound fluorophores in solution. The assays can be multiplexed by differentiating various colored or sized beads. Real time measurement is achievable when a sample is directly required for unlabeled samples. Standard assay steps include incubation of a sample with anti-biomarker antibody coated beads, incubation with biotin or fluorophore-labeled secondary antibody, and detection of fluorescence signals. Fluorescent signals can be developed on bead (by adding streptavidin-fluorophore conjugates for biotinylated secondary antibody) and read out by a bead analyzer. Depending on the anti-biomarker immobilized on the bead surface, a bead-based immunoassay can be a sandwich type or a competitive type immunoassay.

In other specific embodiments, the biomarkers are detected by a protein microarray containing immobilized biomarker-specific antibodies on its surface. The microarray can be used in a “sandwich” assay in which the antibody on the microarray captures a biomarker in the test sample and the captured biomarker is detected by a labeled secondary antibody that specifically binds to the captured biomarker. The secondary antibody can be biotinylated or enzyme-labeled. The detection is achieved by subsequent incubation with a streptavidin-fluorophore conjugate (for fluorescence detection) or an enzyme substrate (for colorimetric detection).

Typically, a microarray assay contains multiple incubation steps, including incubation with the samples and incubation with various reagents (e.g., primary antibodies, secondary antibodies, reporting reagents, etc.). Repeated washes are also needed between the incubation steps. In one embodiment, the microarray assays is performed in a fast assay mode that requires only one or two incubations. It is also conceivable that the formation of a detectable immune complex (e.g., a captured biomarker/anti-biomarker antibody/label complex) may be achieved in a single incubation step by exposing the protein microarray to a mixture of the sample and all the necessary reagents. In one embodiment, the primary and secondary antibodies are the same antibody.

In another specific embodiment, the protein microarray provides a competitive immunoassay. Briefly, a microarray comprising immobilized anti-biomarker antibodies is incubated with a test sample in the presence of a labeled biomarker standard. The labeled biomarker competes with the unlabeled biomarker in the test sample for the binding to the immobilized antigen-specific antibody. In such a competitive setting, an increased concentration of the specific biomarker in the test sample would lead to a decreased binding of the labeled biomarker standard to the immobilized antibody and hence a reduced signal intensity from the label.

The microarray can be processed in manual, semi-automatic or automatic modes. Manual mode refers to manual operations for all assay steps including reagent and sample delivery onto microarrays, sample incubation and microarray washing. Semi-automatic modes refer to manual operation for sample and reagent delivery onto microarray, while incubation and washing steps operate automatically. In an automatic mode, three steps (sample/reagent delivery, incubation and washing) can be controlled by a computer or an integrated breadboard unit with a keypad. For example, the microarray can be processed with a ProteinArray Workstation (PerkinElmer Life Sciences, Boston, Mass.). Scanners by fluorescence, colorimetric and chemiluminescence, can be used to detect microarray signals and capture microarray images. Quantitation of microarray-based assays can also be achieved by other means, such as mass spectrometry and surface plasma resonance. Captured microarray images can be analyzed by stand-alone image analysis software or with image acquisition and analysis software package.

In other specific embodiments, the cancer markers are detected using mass spectrometry (MS) such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, or tandem mass spectrometry (e.g., MS/MS, MS/MS/MS, ESI-MS/MS, etc.).

Mass spectrometry methods are well known in the art and have been used to quantify and/or identify biomarkers, such as proteins. Further, mass spectrometric techniques have been developed that permit at least partial de novo sequencing of isolated proteins. In certain embodiments, a gas phase ion spectrophotometer is used. In other embodiments, laser-desorption/ionization mass spectrometry is used to analyze the sample. Modem laser desorption/ionization mass spectrometry (“LDI-MS”) can be practiced in two main variations: matrix assisted laser desorption/ionization (“MALDI”) mass spectrometry and surface-enhanced laser desorption/ionization (“SELDI”). In MALDI, the analyte is mixed with a solution containing a matrix, and a drop of the liquid is placed on the surface of a substrate. The matrix solution then co-crystallizes with the biological molecules. The substrate is inserted into the mass spectrometer. Laser energy is directed to the substrate surface where it desorbs and ionizes the biological molecules without significantly fragmenting them. In SELDI, the substrate surface is modified so that it is an active participant in the desorption process. In one embodiment, the surface is derivatized with adsorbent and/or capture reagents that selectively bind the protein of interest. In another embodiment, the surface is derivatized with energy absorbing molecules that are not desorbed when struck with the laser. In another embodiment, the surface is derivatized with molecules that bind the protein of interest and that contain a photolytic bond that is broken upon application of the laser. In each of these methods, the derivatizing agent generally is localized to a specific location on the substrate surface where the sample is applied. See, e.g., U.S. Pat. No. 5,719,060 (Hutchens & Yip) and WO 98/59361 (Hutchens & Yip). The two methods can be combined by, for example, using a SELDI affinity surface to capture an analyte and adding matrix-containing liquid to the captured analyte to provide the energy absorbing material.

Detection of the presence of a biomarker will typically involve detection of signal intensity. This, in turn, can reflect the quantity and character of a polypeptide bound to the substrate. For example, in certain embodiments, the signal strength of peak values from spectra of a first sample and a second sample can be compared (e.g., visually, by computer analysis etc.), to determine the relative amounts of particular biomarkers. Software programs can be used to aid in analyzing mass spectra. The mass spectrometers and their techniques are well known to those of skill in the art.

A person skilled in the art understands that any of the components of a mass spectrometer (e.g., desorption source, mass analyzer, detect, etc.) and varied sample preparations can be combined with other suitable components or preparations described herein, or to those known in the art. For example, in some embodiments a control sample may contain heavy atoms (e.g. 13C) thereby permitting the test sample to be mixed with the known control sample in the same mass spectrometry run.

In general, an increase in biomarker protein expression or phosphorylation greater than or equal to at least about 5% when compared to the reference level of protein expression or phosphorylation in patient-matched pre-treatment biopsies or at least about 5% increased biomarker expression or phosphorylation in pre-treatment tumor specimens compared to that in one or multiple control samples (e.g. pre-treatment tumor biopsies of patients that did not show clinical response to PD-1 inhibitors) indicates an increased level of a biomarker, thereby identifying a cancer that will be responsive to treatment comprising PD-1 inhibition. A decrease in protein expression or phosphorylation greater than or equal to about 5% when compared to the reference level of protein expression or phosphorylation obtained from the control sample indicates a decreased level of a biomarker, thereby identifying a cancer that will be responsive and/or is responding (in case of post-treatment tumor biopsy) to treatment comprising PD-1 inhibition.

In other specific embodiments, expression of the biomarker(s) is determined at the mRNA level by quantitative RT-PCR (with or without laser capture microdissection of cancer cells), in situ hybridization, Northern blot, gene microarray, RNAseq, or other methods known to a person of ordinary skill in the art.

RT-PCR involves a single-stranded RNA of a biomarker, which comprises the sequence to be amplified (e.g., an mRNA or portion thereof of a biomarker), and can be incubated in the presence of a reverse transcriptase, two primers, a DNA polymerase, and a mixture of dNTPs suitable for DNA synthesis. mRNA sequences of the biomarkers described herein are well known in the art. During this process, one of the primers anneals to the RNA target and can be extended by the action of the reverse transcriptase, yielding an RNA/cDNA doubled-stranded hybrid. This hybrid can be then denatured and the other primer anneals to the denatured cDNA strand. Once hybridized, the primer can be extended by the action of the DNA polymerase, yielding a double-stranded cDNA, which then serves as the double-stranded target for amplification through PCR. RT-PCR amplification reactions can be carried out with a variety of different reverse transcriptases, and in various embodiments, a thermostable reverse-transcriptions can be used. Quantitative RT-PCR involves amplifying an internal control simultaneously with the biomarker sequence of interest. The internal control is used to normalize the samples. Once normalized, direct comparisons of relative abundance of a specific mRNA can be made across the samples. Commonly used internal controls include, for example, GAPDH, HPRT, actin and cyclophilin.

In general, an increase in biomarker mRNA expression greater than or equal to about a 1-fold increase in expression when compared to the mRNA reference level obtained from the control sample indicates an increased level of a biomarker, thereby identifying a cancer that will be responsive to treatment comprising PD-1 inhibition. A decrease in biomarker mRNA expression greater than or equal to about a 1-fold decrease in expression when compared to the mRNA reference level obtained from the control sample indicates a decreased level of a biomarker, thereby identifying a cancer that will be responsive to treatment comprising PD-1 inhibition.

Methods of the invention can also be used to select or continue a treatment correlated with a good clinical response in a human subject diagnosed with a cancer in which programmed cell death 1 (PD-1) is expressed. A sample obtained from the cancer in question can be evaluated to determine whether there is an increase or decrease in the level of one or more biomarkers of the invention when compared to a reference level or levels from a control sample. Once a cancer is predicted to be responsive to PD-1 inhibition, a treatment correlated with a good clinical response can be selected and administered. A sample obtained from the cancer in question can be evaluated to determine whether PD-1 inhibition is successfully treating the cancer (e.g., the cancer fails to progress following PD-1 inhibition). The treatment can comprise, for example, administering a monoclonal antibody that inhibits PD-1 to the human subject. In specific embodiments, the monoclonal antibody is Pembrolizumab (MK-3475) or Nivolumab (BMS-936558, MDX-1106). Other PD-1 inhibitors known in the art include, but are not limited to, CT-011 (pidilizumab, MDV9300), AMP-224 (PD-1 Inhibitor, B7-DC Fc fusion protein), REGN2810, PDR001, ONO-4538, BGB-A317, MPDL3280A (anti-PD-L1), MPDL3280A (anti-PD-L1), and MEDI4736 (anti-PD-L1). Exemplary dosing of PD-1 inhibitors is shown below in Table 1.

TABLE 1
PD-1 Inhibitor Dosing

| Drug | Indication | Dosing |
|---|---|---|
| Pembrolizumab | Multiple | 200 mg/day every 3 weeks starting day + 14 post |
| | Myeloma | transplant for a total of 9 doses or 180 days. |
| CT-011 | Prostatic | 3 cycles (cycle = 14 days) + CT-011 (3 mg/kg) IV |
| | Neoplasm | infusion. |
| Nivolumab | Melanoma | 1, 3, or 10 mg/kg. |
| Nivolumab | RCC, NSCLC, | 3 mg/kg solution, intravenous, during each 6 week cycle: |
| | Melanoma | every other week (i.e during weeks 1, 3, and 5), Up to 2 |
| | | years. |
| Nivolumab | Melanoma | 6 doses administered every 2 weeks for 12 weeks (Cycle |
| | | 1: Weeks 1, 3, 5, 7, 9, and 11; Cycle 2: Weeks 13, 15, 17, |
| | | 19, 21, and 23) with tumor response assessments at the |
| | | end of each cycle (during Weeks 12 and 24). Level 1: 1 mg/kg |
| | | cohort; Level 2: 3 mg/kg cohort; Level 3: 10 mg/kg |
| | | cohort; Level 4: 3 mg/kg prior ipi gr 0/1/2 cohort; |
| | | Level 5: 3 mg/kg prior ipi gr 3 cohort; Level 6: 3 mg/kg. |
| Pembrolizumab | Multiple | Intravenous infusion at 200 mg every 2 weeks (days 1 |
| | Myeloma | and 14). |
| Nivolumab | Solid tumors | 80 and 240 mg solution intravenously, during each 8 |
| | (NSCLC, | week cycle: every 2 weeks (i.e. during weeks 1, 3, 5, 7), |
| | Melanoma) | up to 96 weeks. |
| Pembrolizumab | oligometastatic | 8 cycles of 3 weekly treatments with MK-3475 (200 mg |
| | breast cancer | per dose). |
| AMP-224 | metastatic | 10 mg/kg on day 1 then every 14 days for a total of 6 |
| | colorectal cancer | doses. |
| Nivolumab | Solid tumors | 3 mg/kg solution intravenously every 2 weeks for 8-96 |
| | | weeks depending on response. |
| Pembrolizumab | Solid tumors | 2 mg/kg q3wks, IV |
| Nivolumab | NSCLC | Two doses of nivolumab will be administered to enrolled |
| | | patients on Day −28 and Day−14 (+/− one day) prior to |
| | | planned surgery on Day 0 or up to +7 days. |
| Nivolumab | RCC | Intravenous infusion, 0.3 mg/kg, 2 mg/kg, 10 mg/kg. |
| | | Every 3 weeks, indefinitely depending on response. |
| CT-011 | RCC | CT-011 at 3 mg/kg IV for 4 cycles of 6 weeks. |
| Pembrolizumab | Melanoma | 2 mg/kg every 3 weeks by intravenous infusion for up to |
| | | 2 years. |
| Pembrolizumab | Melanoma | (200 mg) will be administered intravenously every 3 |
| | | weeks for up to 2 years, beginning on day 1. |
| CT-011 | AML | Infusion given at 6 week intervals for a total of 3 doses. |
| CT-011 | Multiple | 3 doses will be given at 6 week intervals. |
| | Myeloma | |
| CT-011 | Pancreatic Cnacer | 3 mg/kg, intravenous (IV) day 1 of each cycle over 2 |
| | | hours. |
| Nivolumab | Melanoma | Nivolumab 1 or 3 mg/kg IV q2 weeks. |
| Pembrolizumab | CLL, Non- | Pembrolizumab IV over 30 minutes on day 1. Treatment |
| | Hodgkin | repeats every 21 days for up to 12 months i. |
| | Lymphoma | |
| Nivolumab | NSCLC, | 3 mg/kg., treatment cycles are eight weeks each with |
| | Melanoma, | study drug administered once every two weeks. There is |
| | Colorectal, | no limit on the number of cycles of nivolumab. |
| | Ovarian, Head | |
| | and Neck SCC | |
| Nivolumab | Hepatocellular | Nivolumab intravenous solution on specific days. |
| | Cancer | |
| PDR001 | Melanoma, | 2 weeks until patient experiences unacceptable toxicity. |
| | NSCLC, Triple | |
| | Neg Breast | |
| | Cancer | |
| MPDL3280A | Metastaic bladder | 1200 mg dose given by intravenous infusion (IV) on Day |
| | cancer | 1 of 21-day cycles for up to 16 cycles or 12 months |
| | | (whichever comes first). |
| REGN2810 | Solid tumors, | 1 mg/kg, 3 mg/kg, or 10 mg/kg IV q 2 weeks for up to 48 |
| | including | weeks. |
| | cutaneous SCC | |

The present invention is additionally described by way of the following illustrative, non-limiting Examples that provide a better understanding of the present invention and of its many advantages.

### EXAMPLES

The following Examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following Examples do not in any way limit the invention.

The Materials and Methods used to conduct the assays in the following Examples are described in detail herein below.

Tumor cell lines, culture methods and clinical specimens: Human G3361 and C8161 and murine B16-F10 melanoma cell lines were cultured in RPMI-1640 medium (Life Technologies) supplemented with 10% (v/v) fetal bovine serum (Life Technologies) and 1% (v/v) penicillin streptomycin (Life Technologies) and human MKL-1, MKL-2, MS-1, and WaGa Merkel cell carcinoma cell lines in RPMI-1640 medium supplemented with 20% (v/v) fetal bovine serum and 1% (v/v) penicillin streptomycin. Clinical melanoma specimens were obtained from patients in accordance with the Institutional Review Boards of Partners Health Care Research Management, Boston, Mass. and the University of Zurich, Switzerland. Informed consent was obtained from all subjects.

Antibodies: The following antibodies were used for immunohistochemistry (IHC): unconjugated rabbit anti-phospho (p)-protein S6 (Ser235/236), total (t)-S6, p-AKT (Ser473), and 4E-BP1 (Cell Signaling Technology), horseradish peroxidase (HRP)-conjugated anti-rabbit IgG (Vector Laboratories), and biotin-conjugated anti-rabbit IgG (R&D Systems). The following antibodies were used for Western blotting: unconjugated rabbit anti-phospho (p)-protein S6 (Ser235/236), total (t)-S6, p-AKT (Ser473), p-AKT (Ser308), t-AKT (pan), p-ERK1/2 (Thr202/Tyr204), t-ERK, p-PI3K (p85 (Tyr458)/p55 (Tyr199)), t-PI3K (pan), p-PDK1 (Ser241), t-PDK1, p-AMPKα (Thr172), t-AMPKα, p-mTOR (Ser2448), p-PRAS40 (Thr246), p-p70 S6 kinase 1 (Thr389), 4E-BP1, p-eIF4B (Ser422), p-eIF4G (Ser1108), and HRP-conjugated anti-rabbit and anti-mouse (Cell Signaling Technology), mouse anti-β-actin and mouse anti-β-tubulin (BD Biosciences), IRDye 800CW-conjugated goat anti-mouse and goat anti-rabbit, IRDye 680LT-conjugated donkey anti-rabbit and IRDye 680RD-conjugated donkey anti-goat (LI-COR Biosciences).

Immunohistochemical (IHC) staining methods: Tumor biopsy sections were deparaffininzed in xylene and subsequently rehydrated with 100%, 95%, and 75% ethanol and deionized H₂O. Sections were placed in target retrieval solution (Dako) and boiled in a Pascal pressure chamber (Dako) at 125° C. for 30 seconds, 90° C. for 10 seconds, and then cooled down to room temperature. IHC was performed using the two-step peroxidase or alkaline phosphatase method. For peroxidase-based detection of p-S6 and p-AKT, sections were incubated with 1 ug/ml rabbit anti-p-56 ribosomal protein or rabbit anti-p-AKT (Ser473) antibody (Cell Signaling Technology) at 4° C. overnight and then incubated with a 1:200 dilution of goat anti-rabbit IgG peroxidase conjugated antibody (Vector Laboratories) at room temperature for 1 hour. Immunoreactivity was detected using the NovaRed peroxidase substrate (Vector laboratories), according to the manufacturer's protocol. For alkaline phosphatase-based detection of t-SHP2, p-SHP2, p-S6, p-AKT, p-PI3K, p-PDK1, 4E-BP1, or p-eIF-4G, sections were incubated with a 1:200 dilution of t-SHP2, p-SHP2 (Santa Cruz Biotechnology), p-S6, p-AKT, p-PI3K, p-PDK1, 4E-BP1, or p-eIF-4G antibody (Cell Signaling Technology) for 1 hour at room temperature, followed by incubation with a 1:100 dilution of biotin-conjugated secondary antibody (R&D Systems) for 30 minutes at room temperature and a subsequent incubation with a 1:100 dilution of streptavidin-alkaline phosphatase (Roche) for 30 minutes at room temperature. Immunoreactivity was detected using the FAST Red Chromogen System (Biolegend), per the manufacturer's instructions. Nuclear counterstaining (blue) was performed with Meyer's haemalum for both methods.

Imaging and quantification of IHC tumor sections: All images of IHC-stained sections were analyzed with a Nikon microscope (DXM 1200F) and captured using the NIS-Elements software (BR 2.30 Nikon). Expression of p-S6 and 4E-BP1 antigens was graded by n=2-3 independent investigators blinded to the study outcome on a scale of 0-4 (0: no p-S6 expression by melanoma cells; 1: p-S6 expression in 1%-25%; 2: 26%-50%; 3:51%-75%; 4: >75% of melanoma cells). For each slide, at least two areas with the highest numbers of 5-100(+) and MART-1(+) melanoma cells, as determined in serial sections, were selected for analysis.

Western blotting: Protein lysates were extracted from (i) subconfluently plated PD-1 variant (Pdcd1/PDCD1 knockdown (shRNA), overexpressing (OE), or mutant (Y223F, Y248F, Y223F/Y248F)) or PD-1 wildtype murine B16, human G3361 or C8161 melanoma cell lines cultured under serum-starved conditions (0.1% (v/v) FBS for 12 h) in the presence or absence of murine (Biolegend) or human (BioXcell) anti-PD-1 mAb or the respective isotype control mAb (Biolegend/BioXcell) (50 μg/ml, respectively), following subsequent incubation with or without recombinant PD-L1 Ig (R&D Systems) or control Ig (Bethyl Laboratories) (5 μg/ml, 0% (v/v) FBS for 15 minutes, respectively), from (ii) human MKL-1, MKL-2, MS-1, or WaGa Merkel cell carcinoma cell lines cultured in the presence or absence of human anti-PD-1 or isotype control mAb with or without recombinant PD-L1 Ig, PD-L2 Ig, or control Ig, as above, or from (iii) fresh snap frozen Merkel cell carcinoma (MKL-1) xenografts grown in NOD/SCID IL2Rγ (−/−) knockout mice using RIPA buffer supplemented with proteinase (Roche) and phosphatase inhibitor (Roche). Protein concentrations were determined using the BCA protein assay kit (Pierce) according to the manufacturer's protocol. Equal amounts of total protein were resolved by SDS/PAGE and transferred to PVDF membranes. Subsequently, blots were probed overnight at 4° C. with primary antibodies raised against the protein of interest, washed three times for 5 minutes with tris-buffered saline (TBS)/0.1% (v/v) Tween-20 (TBST), incubated with the respective secondary antibody for 1 hour at room temperature, washed three times for 5 minutes with TBST, and developed using enhanced chemoluminescence (Pierce) for horseradish peroxidase (HRP)-conjugated secondary antibodies or analyzed using an Odyssey CLx imaging system (LI-COR Biosciences) for IRDye-conjugated secondary antibodies. Protein expression levels were determined by densitometry (ImageJ, National Institutes of Health).

### Example 1

### Activation of the PD-1 Receptor on Melanoma Cells Promotes Phosphorylation of Mediators of the mTOR, PI3K/AKT and MAPK/ERK Signaling Pathways

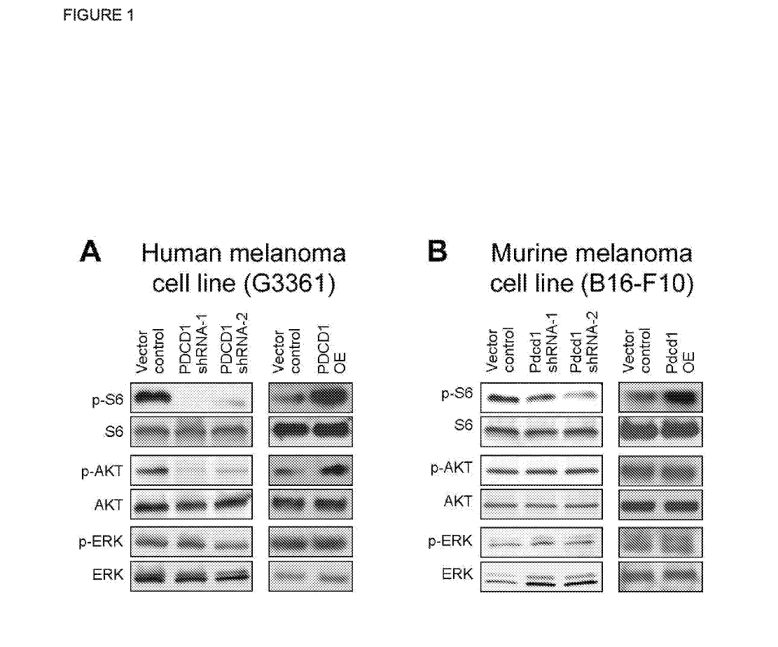

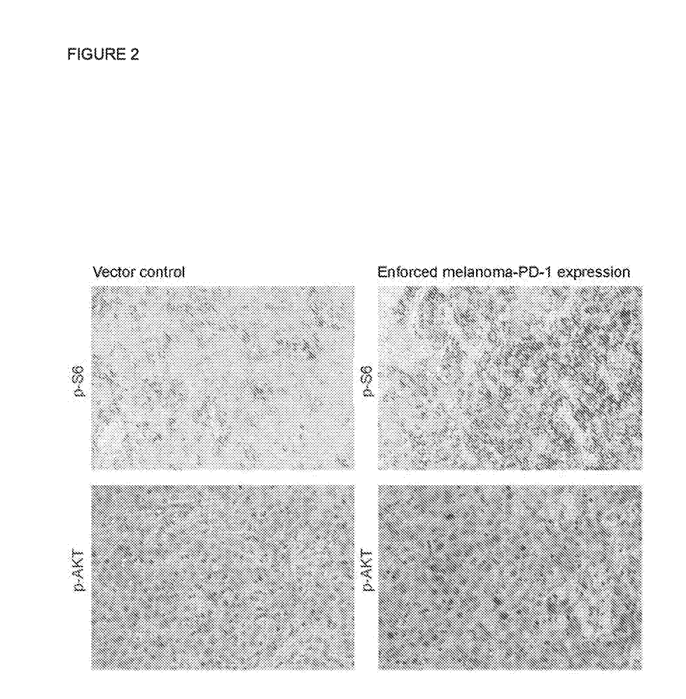

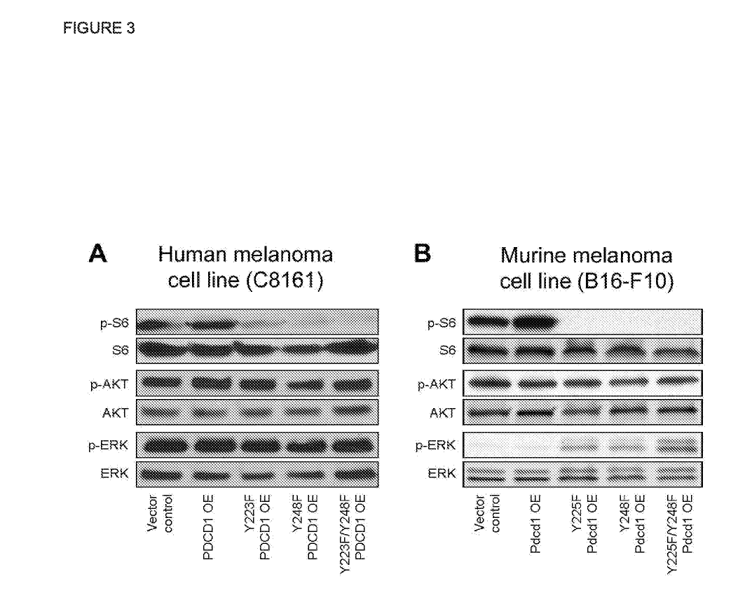

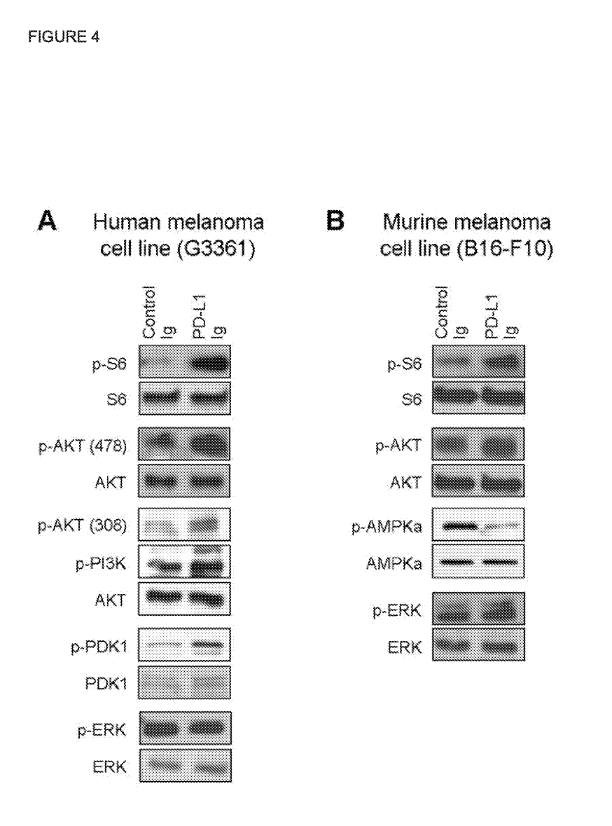

Western blot analysis aimed at identifying the signaling pathways downstream of the PD-1 receptor on melanoma cells revealed that shRNA-mediated knockdown of PD-1 (PDCD1) reduces, and overexpression of PD-1 (PDCD1) enhances, phosphorylation of the mTOR and PI3K/AKT signaling mediators, phospho (p)-S6 and p-AKT, respectively, in human G3361 melanoma cells (FIG. 1A). Similarly, in murine B16-F10 melanoma cells, PD-1 (Pdcd1) knockdown decreases, and PD-1 (Pdcd1) overexpression increases, expression of p-S6, as determined by immunoblotting (FIG. 1B). HRP immunoenzymatic staining of human C8161 melanoma xenografts grown in highly immunocompromised NOD/SCID IL2Rγ (-/-) knockout mice confirmed enhanced expression of p-S6 and p-AKT in PD-1-overexpressing (enforced melanoma PD-1 expression) compared to vector control-transduced melanomas (FIG. 2). Mutagenesis of signaling motifs (ITIM, disrupted by Y223F (human PD-1) or Y225F (murine PD-1) mutation; ITSM, disrupted by Y248F mutation; ITSM and ITIM, disrupted by Y223F/Y248F or Y225F/Y248F double-mutation) within the cytoplasmic tail of the melanoma-PD-1 receptor, which are known to bind/recruit and activate the phosphatase SHP2 (also known as PTPN11), resulted in substantially reduced p-S6 expression levels compared to wildtype PD-1 overexpression of vector control human C8161 (FIG. 3A) and murine B16-F10 melanoma cells (FIG. 3B), thereby further confirming p-S6 as a signaling mediator downstream of the melanoma-PD-1 receptor. Treatment of human wildtype PD-1-expressing G3361 melanoma cells with a recombinant PD-1 ligand 1 (PD-L1) Fc-fusion protein (PD-L1 Ig), known to elicit changes in PD-1 receptor signaling in T-cells, resulted in enhanced expression of the mTOR and/or PI3K/AKT signaling mediators, p-S6, p-AKT (serins 478 and 308), p-PDK1, and p-PI3K compared to control Ig treatment (FIG. 4A). In murine B16-F10 melanoma cells, melanoma-PD-1 engagement by PD-L1 Ig enhanced phosphorylation of S6, AKT, and the MAPK signaling mediator, ERK, and reduced phosphorylation of AMPKa, compared to control-treated cultures (FIG. 4B). Together, these findings identify members of the mTOR, PI3K/AKT, and MAPK/ERK signaling pathways as biomarkers that correspond to tumor cell-intrinsic PD-1 pathway activity, including p-SHP2, in melanoma cells.

### Example 2

### Antibody-Mediated PD-1 Blockade Inhibits Phosphorylation of mTOR, PI3K/AKT and/or MAPK/ERK Signaling Mediators in Melanoma Cells

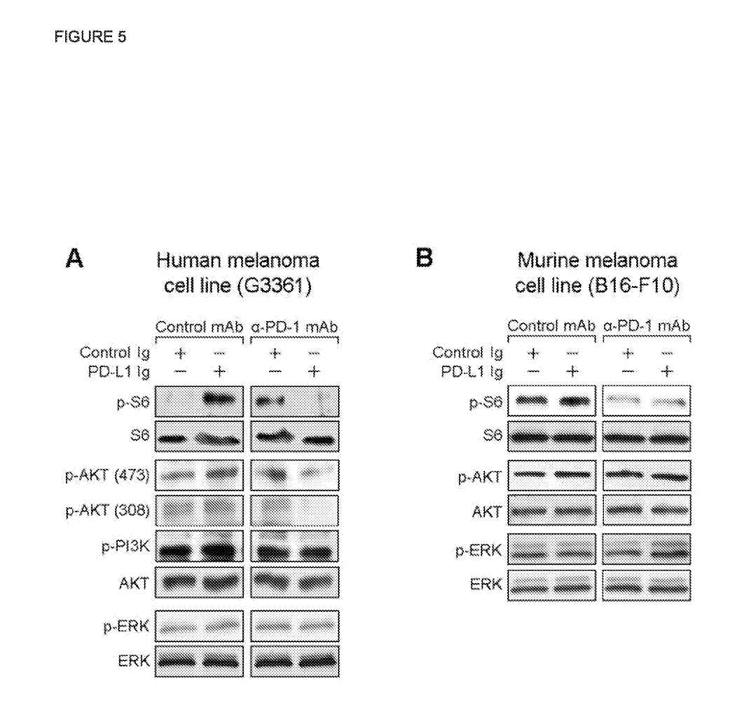

Next, the effects of antibody-mediated PD-1 blockade on signaling pathways downstream of the PD-1 receptor were examined in melanoma cells. Treatment of G3361 melanoma cultures with a PD-1 blocking but not isotype control antibody inhibited PD-L1 Ig-dependent phosphorylation of S6, AKT (serins 478 and 308), and PI3K (FIG. 5A). In murine B16-F10 melanoma cultures, antibody-mediated PD-1 blockade inhibited phosphorylation of S6 (FIG. 5B). Together, these findings identify mTOR and PI3K/AKT, signaling pathway members as biomarkers for (i) monitoring and (ii) predicting response to PD-1 pathway blockade in melanoma. Additionally, these biomarkers can serve as tools to (iii) design rational combination therapies on a patient-by-patient basis, according to the oncogenic pathways affected (or not affected) by PD-1 blockade in a given melanoma sample, including patient-derived melanoma cells.

### Example 3

### Activation of the Phosphatase, SHP2 (PTPN11), in Pre-Treatment Tumor Biopsies Correlates with Response to Clinical PD-1 Pathway Inhibitors

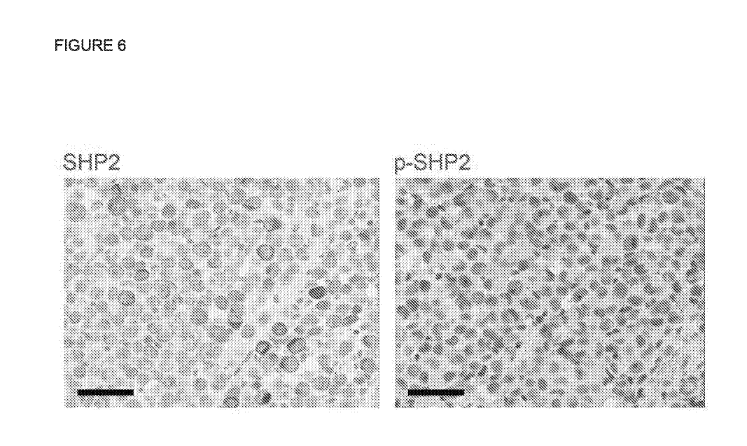

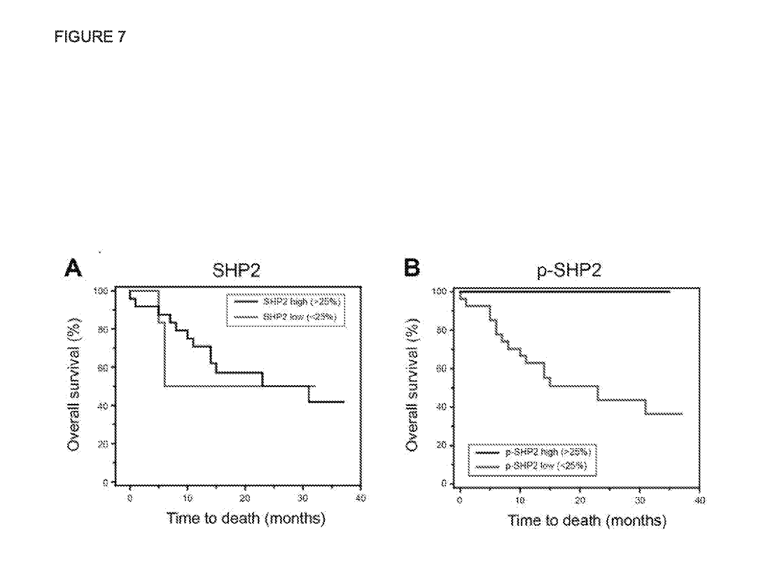

Immunohistochemical staining of pre-treatment tumor biopsies of melanoma patients undergoing anti-PD-1 therapy revealed subgroups of patients with high expression (greater than 25% of melanoma cells) and low expression (less than 25% of melanoma cells) of SHP2 and its activated form, phosphorylated (p-) SHP2 (representative IHC staining is illustrated in FIG. 6). Kaplan Meier analyses revealed no significant difference in overall survival in response to anti-PD-1 therapy between patients with high vs. low total (t-) SHP2 expression (FIG. 7A). However, 100% of patients demonstrating >25% p-SHP2 expression in melanoma biopsies before treatment with therapeutic PD-1 antibodies (Nivolumab or Pemprolizumab) were still alive 35 months after initiation of treatment compared to less than 40% of patients with low p-SHP2 expression (FIG. 7B).

### Example 4

### mTOR and/or PI3K/AKT Pathway Activation in Pre-Treatment Tumor Biopsies Correlates with Response to Clinical PD-1 Pathway Inhibitors

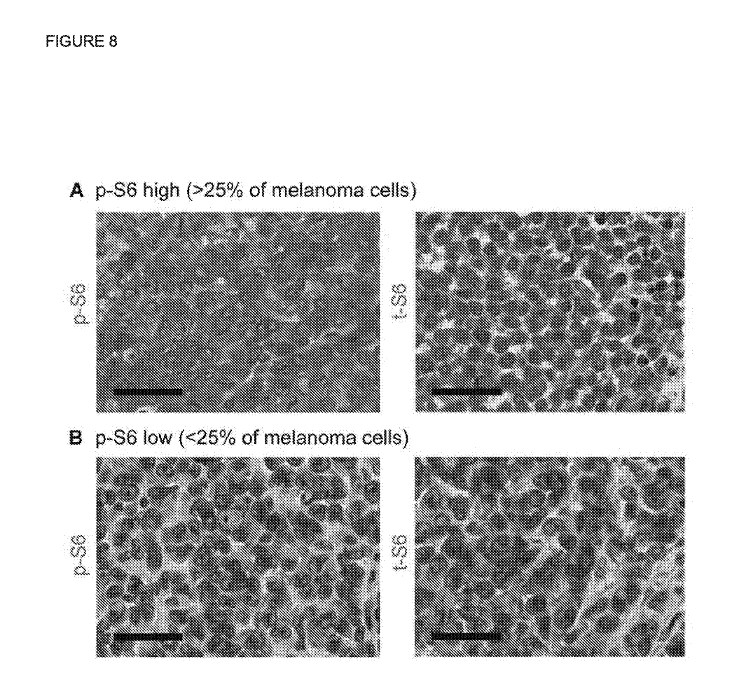

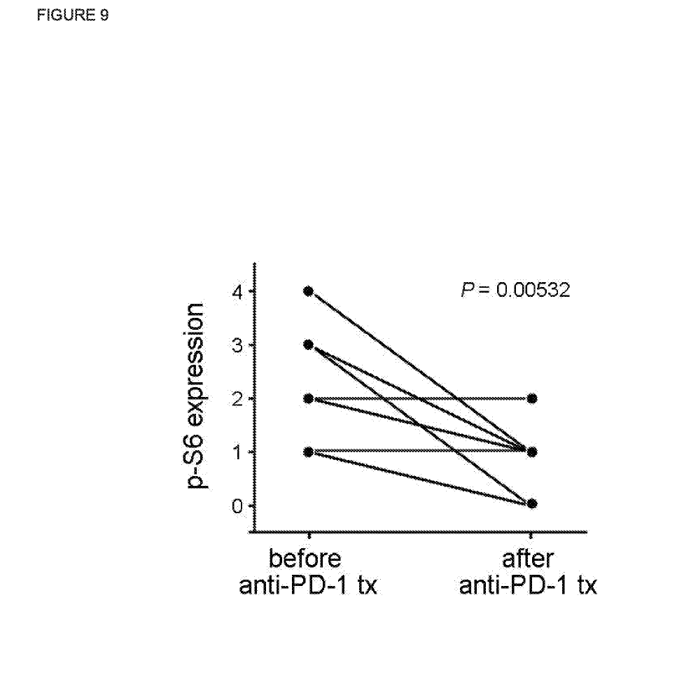

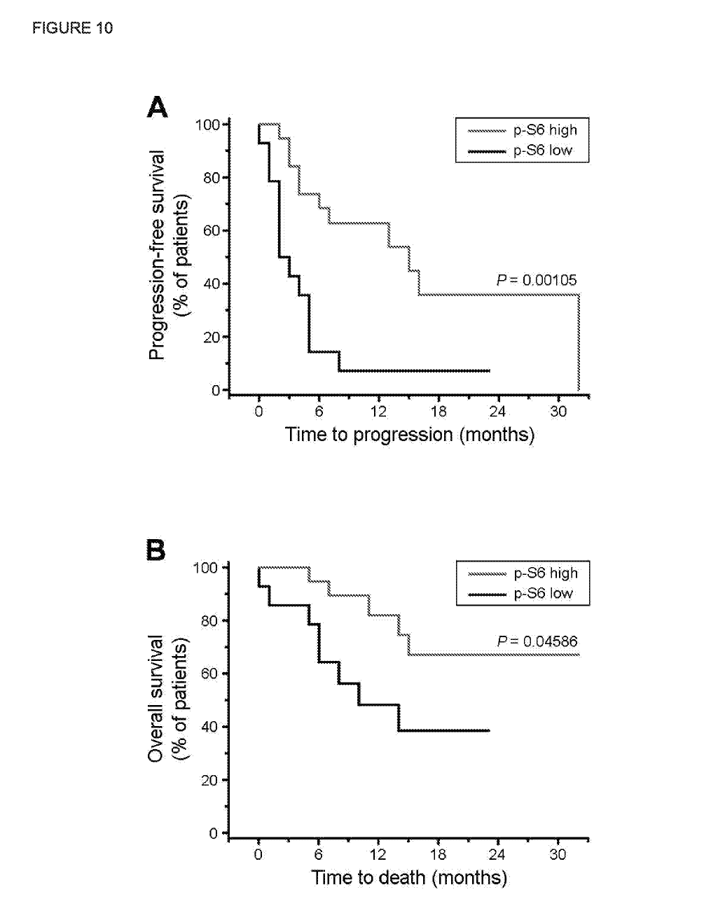

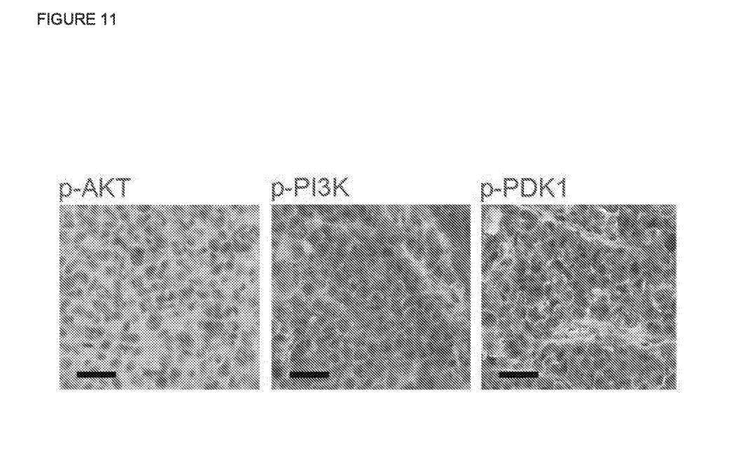

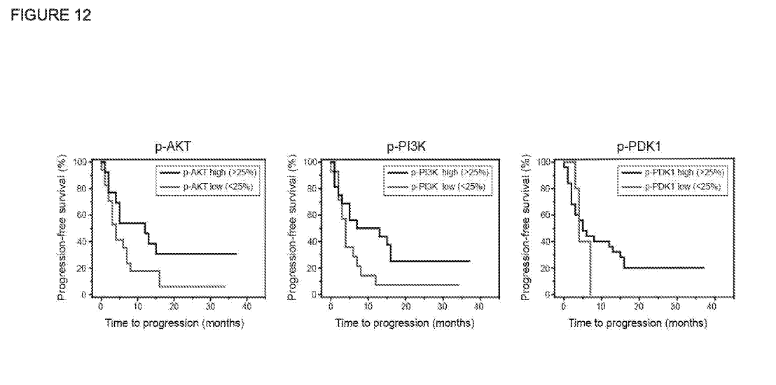

Immunohistochemical staining of pre-treatment tumor biopsies of melanoma patients undergoing PD-1 inhibitor trials revealed high expression of p-S6 (greater than 25% of melanoma cells) in the majority of patients with good clinical response (mean progression-free survival: 17 months and mean overall survival: 25.1 months, FIG. 8A) and low p-S6 expression (less than 25% of melanoma cells) in patients with poor clinical response to PD-1 antibody treatment (mean progression-free survival: 4.5 months and mean overall survival: 13.0 months, FIG. 8B). Assessment of melanoma-p-S6 expression in pre-treatment vs. post-treatment tumor biopsies obtained from n=11 melanoma patients undergoing anti-PD-1 theapy revealed significantly decreased p-S6 expression in melanoma biospecimens sampled post PD-1 therapy compared to patient-matched pre-treatment biopsies (FIG. 9). Kaplan-Meier analyses revealed that patients (n=34) demonstrating >25% melanoma-p-S6 expression in pre-treatment tumor biopsies showed a >3-fold increase in progression-free (FIG. 10A) and significantly enhanced overall survival (FIG. 10B) in response to clinical PD-1 inhibitor treatment (Nivolumab or Pembrolizumab) compared to patients with low (less than 25%) melanoma-p-S6 expression. Additionally, patients with high expression (>25% of melanoma cells) of the PI3K pathway members, p-AKT, p-PI3K, or p-PDK1, in pre-treatment biopsies (representative IHC staining is illustrated in FIG. 11) showed an increase in progression-free survival in response to anti-PD-1 therapy, compared to patients with low (<25%) p-AKT, p-PI3K, or p-PDK1 expression (FIG. 12).

### Example 5

### Activation of the PD-1 Receptor on Melanoma Cells Results in Enhanced Phosphorylation of Cap-Dependent Translation Initiation Factors

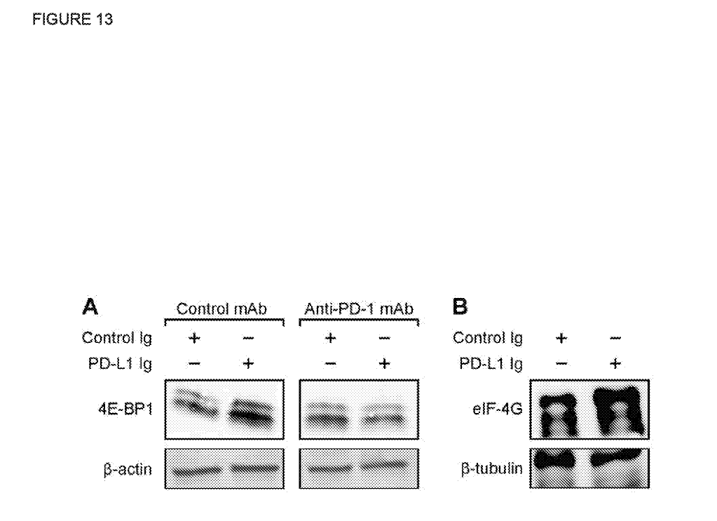

Treatment of human G3361 melanoma cultures with PD-L1 Ig increased expression of eukaryotic translation initiation factor 4E binding protein 1 (4E-BP1) (FIG. 13A) and eIF-4G (FIG. 13B) compared to control Ig treatment, as determined by Western blot analysis. Moreover, antibody-mediated PD-1 blockade inhibited PD-L1 Ig-induced expression of 4E-BP1 (FIG. 13A). Together, these fmdings identify cap-dependent translation initiation factors as biomarkers for (i) monitoring and (ii) predicting response to PD-1 pathway blockade.

### Example 6

### High expression of 4E-BP1 and eIF-4G in Pre-Treatment Tumor Biopsies Correlates with Clinical Response to PD-1 Pathway Inhibitors

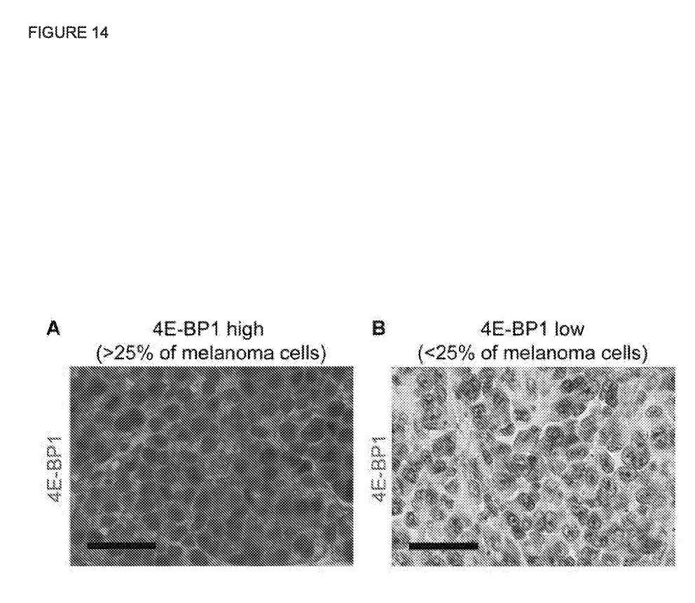

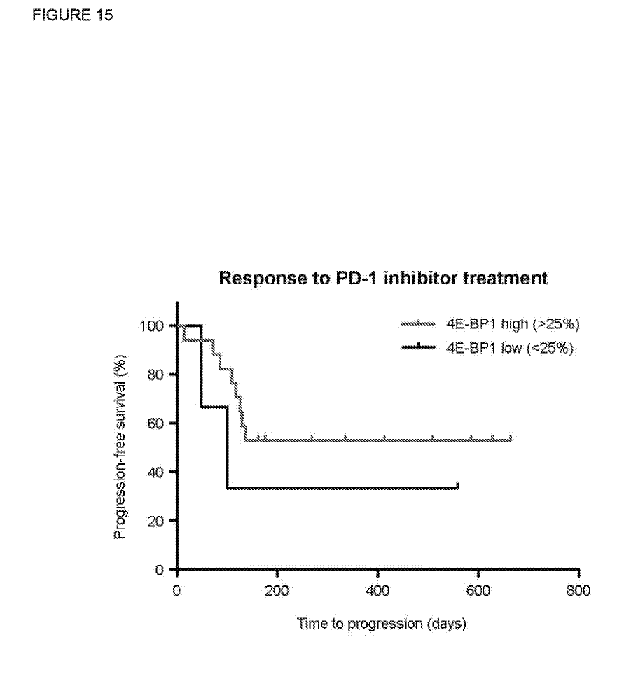

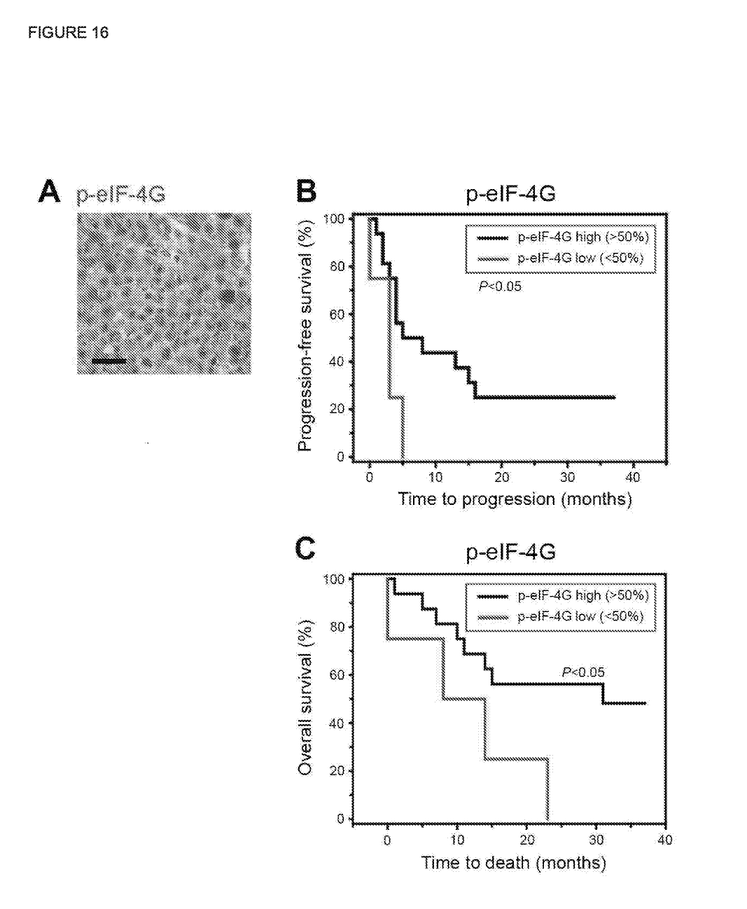

IHC staining of pre-treatment tumor biopsies of melanoma patients undergoing PD-1 inhibitor trials revealed high expression of 4E-BP1 (greater than 25%) in the majority of patients with good clinical response (long progression-free and overall survival, FIG. 14A) and low 4E-BP1 expression (less than 25%) in patients with poor clinical response to PD-1 antibody treatment (short progression-free and overall survival, FIG. 14B). Kaplan-Meier analyses revealed that patients demonstrating greater than 25% 4E-BP1 expression in pre-treatment biopsies had a significantly increased progression-free (FIG. 15) survival in response to clinical PD-1 inhibitor treatment (Nivolumab or Pembrolizumab) compared to patients with low (less than 25%) 4E-BP1 expression. Additionally, patients with greater than 50% phosphorylated (p-) eIF-4G expression in pre-treatment biopsies (representative IHC staining depicted in FIG. 16A) showed a significant increase in progression-free (FIG. 16B) and overall survival (FIG. 16C) compared to patients with less than 50% p-eIF-4G expression, as determined by Kaplan Meier analysis.

### Example 7

### Activation of the PD-1 Receptor on Merkel Cell Carcinoma Cells Triggers Phosphorylation of mTOR Signaling Mediators

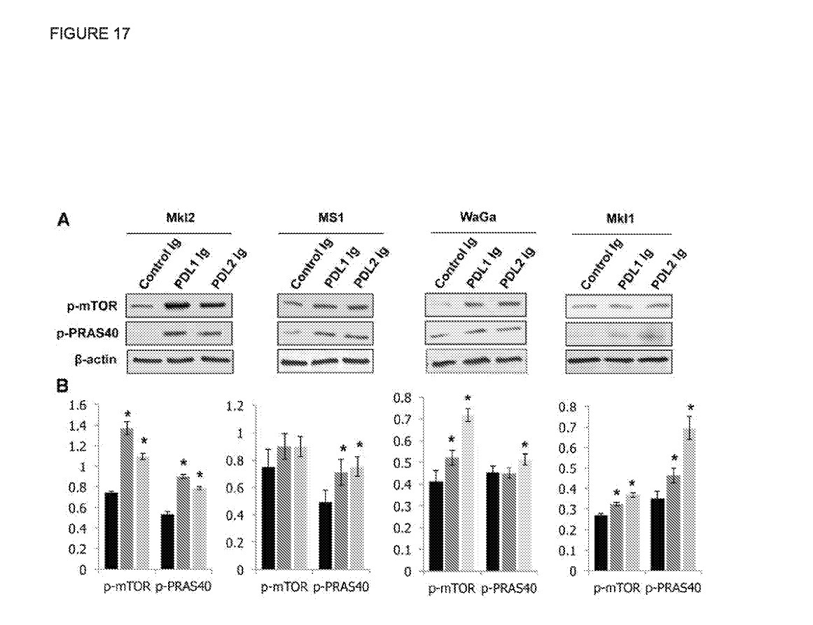

In addition to being expressed by cells of the hematopoietic lineage and melanoma cells, the PD-1 receptor is also expressed by Merkel cell carcinoma (MCC) cells. Treatment of human MCC cell lines, MKL-1, MKL-2, MS-1, and WaGa with recombinant PD-1 receptor ligands, PD-L1 Ig or PD-L2 Ig, resulted in enhanced phosphorylation of mTOR and PRAS40, as determined by Western blotting (FIG. 17A) and subsequent densitometry of protein band intensities (FIG. 17B). Together, these findings identify members of the mTOR signaling pathway as biomarkers of PD-1 pathway activity in MCC cells.

### Examples 8

### Antibody-Mediated PD-1 Blockade Inhibits Phosphorylation of mTOR Signaling Mediators in Merkel Cell Carcinoma Cells and Experimental tumors

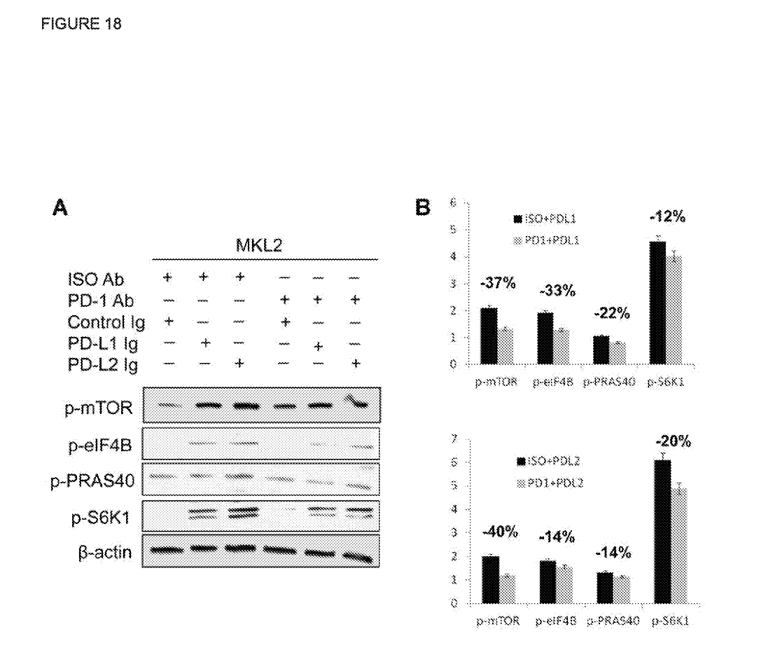

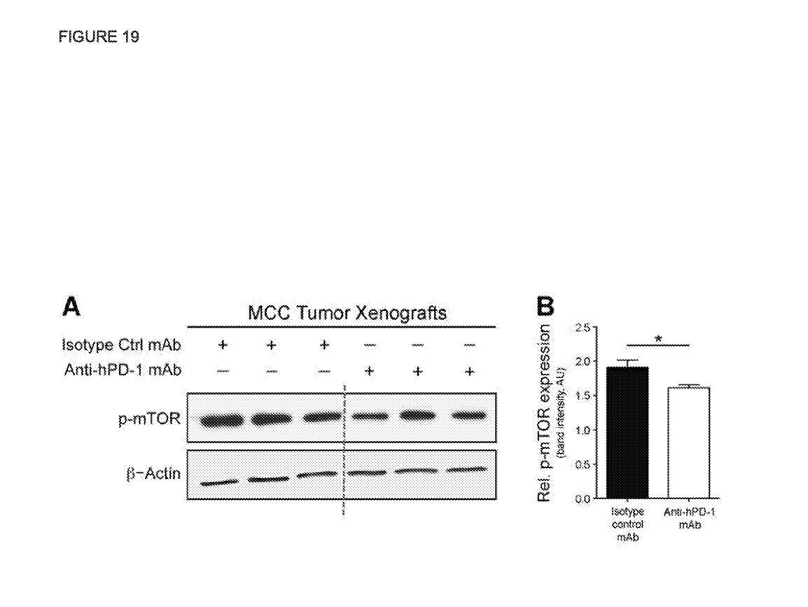

Next, the effects of antibody-mediated PD-1 blockade on signaling pathways downstream of the PD-1 receptor in MCC cells were examined. Treatment of MKL-2 cultures with a PD-1 blocking but not isotype control antibody inhibited PD-L1 Ig- and PD-L2-Ig-dependent phosphorylation of mTOR, PRAS40, as well as S6 kinase 1, and the translation initiation factor eIF-4B, as determined by Western blotting (FIG. 18A). Densitometric analysis of protein band intensities revealed a PD-1 antibody-mediated reduction in PD-L1 Ig-induced phosphoproteins ranging from 12-37% and in PD-L2 Ig-induced phosphoproteins ranging from 14-40% compared to isotype control treatment (FIG. 18B). Consistent with these findings, treatment of MCC tumor xenograft-bearing NOD/SCID IL2Rγ (−/−) knockout mice with a PD-1 blocking antibody resulted in significant tumor growth inhibition compared to isotype control treatment (not illustrated) concomitant with significantly reduced p-mTOR levels in PD-1 antibody- vs. isotype control-treated MCC (MKL-1) tumor xenografts (FIGS. 19A and 19B). Together, these fmdings identify mTOR pathway members, p-mTOR, p-PRAS40, p-S6K1, and translation initiation factor, eIF-4B, as biomarkers for monitoring and predicting response to PD-1 pathway blockade.

### REFERENCES

All patents, patent applications and publications mentioned in this specification are herein incorporated by reference to the same extent as if each independent patent and publication was specifically and individually indicated to be incorporated by reference.

1. A method of selecting a treatment correlated with a good clinical response in a human subject diagnosed with a cancer in which PD-1 is expressed, said method comprising the steps of:
a) determining whether there is an increase in the level of AMP-activated protein kinase alpha and/or a decrease in the level of any one of Ribosomal protein S6, AKT, Phosphoinositide 3-kinase, Phosphoinositide-dependent protein kinase 1, Extracellular signal regulated kinase, Translation initiation factor 4E binding protein 1, Eukaryotic translation initiation factor 4B, Eukaryotic translation initiation factor 4G, mammalian target of rapamycin, PRAS40, S6 kinase 1, and src homology 2-containing protein-tyrosine-phosphatase, or any combination thereof, in a sample of the cancer, wherein said level or levels are compared to a reference level or levels; and
b) selecting a treatment comprising PD-1 inhibition to administer to the human subject, thereby selecting a treatment correlated with a good clinical response in the human subject.

2. The method of claim 1, wherein the level is an amount of nucleic acid or protein expression.

3. The method of claim 1, where in the level is an amount of phosphorylation of any one of Ribosomal protein S6, AKT, Phosphoinositide 3-kinase, Phosphoinositide-dependent protein kinase 1, Extracellular signal regulated kinase, Eukaryotic translation initiation factor 4B, Eukaryotic translation initiation factor 4G, mammalian target of rapamycin, PRAS40, S6 kinase 1, or src homology 2-containing protein-tyrosine-phosphatase, or any combination thereof.

4. The method of claim 1, wherein the cancer is melanoma, merkel cell carcinoma, lung cancer, renal cancer, hodkins lymphoma, glioblastoma, hepatocellular carcinoma, colorectal carcinoma, bladder cancer, pancreatic cancer, head and neck cancer, squamous cell carcinoma, diffuse large B-cell lymphoma, prostate cancer, or any cancer subtype showing microsatellite instability or breast cancer.

5. The method of claim 1, wherein the treatment comprises administering a monoclonal antibody, pharmacologic agent, biologic agent, or medicinal product that inhibits PD-1 to the human subject.

6. The method of claim 5, wherein the monoclonal antibody is Pembrolizumab, Nivolumab, CT-011, AMP-244 or PDR001.

7. A method of identifying a PD-1 expressing cancer in a human subject that would fail to progress in response to PD-1 inhibition, said method comprising the steps of:
a) determining whether there is an increase in the level of total and/or phosphorylated AMP-activated protein kinase alpha and/or a decrease in the level of any one of total and/or phosphorylated Ribosomal protein S6, AKT, Phosphoinositide 3-kinase, Phosphoinositide-dependent protein kinase 1, Extracellular signal regulated kinase, Translation initiation factor 4E binding protein 1, Eukaryotic translation initiation factor 4B, Eukaryotic translation initiation factor 4G, mammalian target of rapamycin, PRAS40, S6 kinase 1, and src homology 2-containing protein-tyrosine-phosphatase, or any combination thereof, in a sample of the cancer, wherein said level or levels are compared to a reference level or levels; and
b) characterizing the cancer as one that would fail to progress in response to PD-1 inhibition.

8. The method of claim 7, wherein the level is an amount of nucleic acid or protein expression.

9. The method of claim 7, where in the level is an amount of protein phosphorylation of any one of Ribosomal protein S6, AKT, Phosphoinositide 3-kinase, Phosphoinositide-dependent protein kinase 1, Extracellular signal regulated kinase, Eukaryotic translation initiation factor 4B, Eukaryotic translation initiation factor 4G, mammalian target of rapamycin, PRAS40, S6 kinase 1, or src homology 2-containing protein-tyrosine-phosphatase, or any combination thereof.

10. The method of claim 7, wherein the cancer is melanoma, merkel cell carcinoma, lung cancer, renal cancer, hodkins lymphoma, glioblastoma, hepatocellular carcinoma, colorectal carcinoma, bladder cancer, pancreatic cancer, head and neck cancer, squamous cell carcinoma, diffuse large B-cell lymphoma, prostate cancer, or any cancer subtype showing microsatellite instability or breast cancer.

11. The method of claim 7, wherein a cancer that fails to progress is decreased in size or severity following PD-1 inhibition.

12. The method of claim 7, wherein the subject is undergoing therapy comprising PD-1 inhibition.

13. The method of claim 12, wherein the reference level is obtained from a cancer that that continues to progress following PD-1 inhibition.

14. A method of treating a human subject diagnosed with a cancer in which PD-1 is expressed, said method comprising the steps of:
a) administering a treatment comprising an inhibitor of PD-1 to the human subject;
b) determining whether there is an increase in the level of AMP-activated protein kinase alpha and/or a decrease in the level of any one of Ribosomal protein S6, AKT, Phosphoinositide 3-kinase, Phosphoinositide-dependent protein kinase 1, Extracellular signal regulated kinase, Translation initiation factor 4E binding protein 1, Eukaryotic translation initiation factor 4B, Eukaryotic translation initiation factor 4G, mammalian target of rapamycin, PRAS40, S6 kinase 1, and src homology 2-containing protein-tyrosine-phosphatase, or any combination thereof, in a sample of the cancer, wherein said level or levels are compared to a reference level or levels; and
c) continuing to administer a treatment comprising an inhibitor of PD-1 to the human subject, thereby treating the human subject.

15. The method of claim 14, wherein the reference level is obtained from a cancer that that continues to progress following PD-1 inhibition.

16. A method of selecting a treatment correlated with a good clinical response in a human subject diagnosed with a cancer in which PD-1 is expressed, said method comprising the steps of:
a) determining whether there is at least about 5% expression of AMP-activated protein kinase alpha and/or a decrease in the level of any one of Ribosomal protein S6, AKT, Phosphoinositide 3-kinase, Phosphoinositide-dependent protein kinase 1, Extracellular signal regulated kinase, Translation initiation factor 4E binding protein 1, Eukaryotic translation initiation factor 4B, Eukaryotic translation initiation factor 4G, mammalian target of rapamycin, PRAS40, S6 kinase 1, src homology 2-containing protein-tyrosine-phosphatase, or any combination thereof, in a sample of the cancer; and
b) selecting a treatment comprising PD-1 inhibition to administer to the human subject, thereby selecting a treatment correlated with a good clinical response in the human subject.

17. The method of claim 14, wherein the cancer in which PD-1 is expressed comprises subsets of cells that express PD-1 and other cells that do not express PD-1 or detectable levels of PD-1.


SEQUENCE LISTING (28 sequences)

SEQ ID NO: 1 - DNA, 845 bp, Homo sapiens
cctcggaggcgttcagctgcttcaagatgaagctgaacatctccttcccagccactggctgccagaaactcattgaagtggacgatgaacgcaaacttcgtactttctatgagaagcgtatggccacagaagttgctgctgacgctctgggtgaagaatggaagggttatgtggtccgaatcagtggtgggaacgacaaacaaggtttccccatgaagcagggtgtcttgacccatggccgtgtccgcctgctactgagtaaggggcattcctgttacagaccaaggagaactggagaaagaaagagaaaatcagttcgtggttgcattgtggatgcaaatctgagcgttctcaacttggttattgtaaaaaaaggagagaaggatattcctggactgactgatactacagtgcctcgccgcctgggccccaaaagagctagcagaatccgcaaacttttcaatctctctaaagaagatgatgtccgccagtatgttgtaagaaagcccttaaataaagaaggtaagaaacctaggaccaaagcacccaagattcagcgtcttgttactccacgtgtcctgcagcacaaacggcggcgtattgctctgaagaagcagcgtaccaagaaaaataaagaagaggctgcagaatatgctaaacttttggccaagagaatgaaggaggctaaggagaagcgccaggaacaaattgcgaagagacgcagactttcctctctgcgagcttctacttctaagtctgaatccagtcagaaataagattttttgagtaacaaataaataagatcagactctgaaaaaaaaaaaaaaaaaaaaaaaaaaaaaaaa

SEQ ID NO: 2 - protein, 249 aa, Homo sapiens
MKLNISFPATGCQKLIEVDDERKLRTFYEKRMATEVAADALGEEWKGYVVRISGGNDKQGFPMKQGVLTHGRVRLLLSKGHSCYRPRRTGERKRKSVRGCIVDANLSVLNLVIVKKGEKDIPGLTDTTVPRRLGPKRASRIRKLFNLSKEDDVRQYVVRKPLNKEGKKPRTKAPKIQRLVTPRVLQHKRRRIALKKQRTKKNKEEAAEYAKLLAKRMKEAKEKRQEQIAKRRRLSSLRASTSKSESSQK

SEQ ID NO: 3 - DNA, 2610 bp, Homo sapiens
atcctgggacagggcacagggccatctgtcaccaggggcttagggaaggccgagccagcctgggtcaaagaagtcaaaggggctgcctggaggaggcagcctgtcagctggtgcatcagaggctgtggccaggccagctgggctcggggagcgccagcctgagaggagcgcgtgagcgtcgcgggagcctcgggcaccatgagcgacgtggctattgtgaaggagggttggctgcacaaacgaggggagtacatcaagacctggcggccacgctacttcctcctcaagaatgatggcaccttcattggctacaaggagcggccgcaggatgtggaccaacgtgaggctcccctcaacaacttctctgtggcgcagtgccagctgatgaagacggagcggccccggcccaacaccttcatcatccgctgcctgcagtggaccactgtcatcgaacgcaccttccatgtggagactcctgaggagcgggaggagtggacaaccgccatccagactgtggctgacggcctcaagaagcaggaggaggaggagatggacttccggtcgggctcacccagtgacaactcaggggctgaagagatggaggtgtccctggccaagcccaagcaccgcgtgaccatgaacgagtttgagtacctgaagctgctgggcaagggcactttcggcaaggtgatcctggtgaaggagaaggccacaggccgctactacgccatgaagatcctcaagaaggaagtcatcgtggccaaggacgaggtggcccacacactcaccgagaaccgcgtcctgcagaactccaggcaccccttcctcacagccctgaagtactctttccagacccacgaccgcctctgctttgtcatggagtacgccaacgggggcgagctgttcttccacctgtcccgggaacgtgtgttctccgaggaccgggcccgcttctatggcgctgagattgtgtcagccctggactacctgcactcggagaagaacgtggtgtaccgggacctcaagctggagaacctcatgctggacaaggacgggcacattaagatcacagacttcgggctgtgcaaggaggggatcaaggacggtgccaccatgaagaccttttgcggcacacctgagtacctggcccccgaggtgctggaggacaatgactacggccgtgcagtggactggtgggggctgggcgtggtcatgtacgagatgatgtgcggtcgcctgcccttctacaaccaggaccatgagaagctttttgagctcatcctcatggaggagatccgcttcccgcgcacgcttggtcccgaggccaagtccttgctttcagggctgctcaagaaggaccccaagcagaggcttggcgggggctccgaggacgccaaggagatcatgcagcatcgcttctttgccggtatcgtgtggcagcacgtgtacgagaagaagctcagcccacccttcaagccccaggtcacgtcggagactgacaccaggtattttgatgaggagttcacggcccagatgatcaccatcacaccacctgaccaagatgacagcatggagtgtgtggacagcgagcgcaggccccacttcccccagttctcctactcggccagcagcacggcctgaggcggcggtggactgcgctggacgatagcttggagggatggagaggcggcctcgtgccatgatctgtatttaatggtttttatttctcgggtgcatttgagagaagccacgctgtcctctcgagcccagatggaaagacgtttttgtgctgtgggcagcaccctcccccgcagcggggtagggaagaaaactatcctgcgggttttaatttatttcatccagtttgttctccgggtgtggcctcagccctcagaacaatccgattcacgtagggaaatgttaaggacttctacagctatgcgcaatgtggcattggggggccgggcaggtcctgcccatgtgtcccctcactctgtcagccagccgccctgggctgtctgtcaccagctatctgtcatctctctggggccctgggcctcagttcaacctggtggcaccagatgcaacctcactatggtatgctggccagcaccctctcctgggggtggcaggcacacagcagccccccagcactaaggccgtgtctctgaggacgtcatcggaggctgggcccctgggatgggaccagggatgggggatgggccagggtttacccagtgggacagaggagcaaggtttaaatttgttattgtgtattatgttgttcaaatgcattttgggggtttttaatctttgtgacaggaaagccctcccccttccccttctgtgtcacagttcttggtgactgtcccaccggagcctccccctcagatgatctctccacggtagcacttgaccttttcgacgcttaacctttccgctgtcgccccaggccctccctgactccctgtgggggtggccatccctgggcccctccacgcctcctggccagacgctgccgctgccgctgcaccacggcgtttttttacaacattcaactttagtatttttactattataatataatatggaaccttccctccaaattct

SEQ ID NO: 4 - protein, 480 aa, Homo sapiens
MSDVAIVKEGWLHKRGEYIKTWRPRYFLLKNDGTFIGYKERPQDVDQREAPLNNFSVAQCQLMKTERPRPNTFIIRCLQWTTVIERTFHVETPEEREEWTTAIQTVADGLKKQEEEEMDFRSGSPSDNSGAEEMEVSLAKPKHRVTMNEFEYLKLLGKGTFGKVILVKEKATGRYYAMKILKKEVIVAKDEVAHTLTENRVLQNSRHPFLTALKYSFQTHDRLCFVMEYANGGELFFHLSRERVFSEDRARFYGAEIVSALDYLHSEKNVVYRDLKLENLMLDKDGHIKITDFGLCKEGIKDGATMKTFCGTPEYLAPEVLEDNDYGRAVDWWGLGVVMYEMMCGRLPFYNQDHEKLFELILMEEIRFPRTLGPEAKSLLSGLLKKDPKQRLGGGSEDAKEIMQHRFFAGIVWQHVYEKKLSPPFKPQVTSETDTRYFDEEFTAQMITITPPDQDDSMECVDSERRPHFPQFSYSASSTA

SEQ ID NO: 5 - protein, 724 aa, Homo sapiens
MSAEGYQYRALYDYKKEREEDIDLHLGDILTVNKGSLVALGFSDGQEARPEEIGWLNGYNETTGERGDFPGTYVEYIGRKKISPPTPKPRPPRPLPVAPGSSKTEADVEQQALTLPDLAEQFAPPDIAPPLLIKLVEAIEKKGLECSTLYRTQSSSNLAELRQLLDCDTPSVDLEMIDVHVLADAFKRYLLDLPNPVIPAAVYSEMISLAPEVQSSEEYIQLLKKLIRSPSIPHQYWLTLQYLLKHFFKLSQTSSKNLLNARVLSEIFSPMLFRFSAASSDNTENLIKVIEILISTEWNERQPAPALPPKPPKPTTVANNGMNNNMSLQDAEWYWGDISREEVNEKLRDTADGTFLVRDASTKMHGDYTLTLRKGGNNKLIKIFHRDGKYGFSDPLTFSSVVELINHYRNESLAQYNPKLDVKLLYPVSKYQQDQVVKEDNIEAVGKKLHEYNTQFQEKSREYDRLYEEYTRTSQEIQMKRTAIEAFNETIKIFEEQCQTQERYSKEYIEKFKREGNEKEIQRIMHNYDKLKSRISEIIDSRRRLEEDLKKQAAEYREIDKRMNSIKPDLIQLRKTRDQYLMWLTQKGVRQKKLNEWLGNENTEDQYSLVEDDEDLPHHDEKTWNVGSSNRNKAENLLRGKRDGTFLVRESSKQGCYACSVVVDGEVKHCVINKTATGYGFAEPYNLYSSLKELVLHYQHTSLVQHNDSLNVTLAYPVYAQQRR

SEQ ID NO: 6 - DNA, 2430 bp, Homo sapiens
agagagagaaactccgggacactcccgatggcaccttcctagtccgagatgcttctagcaagatccagggcgagtacacgctgaccctcaggaaaggcgggaacaataagctgatcaaggtcttccaccgagatgggcactatggcttctcagagccactcaccttctgctccgttgtggacctcatcaatcactaccgccacgagtctctggcccagtacaatgccaagctggacacacggctcctctaccctgtgtccaaataccagcaggaccagattgtcaaggaggacagcgtggaggcagtgggcgcccagcttaaggtctatcaccagcagtaccaggacaagagccgcgagtatgaccagctttatgaagagtacacacggacctcccaggagctgcagatgaagcgtactgcaattgaggccttcaatgagactatcaagatctttgaagagcagggccagactcaagagaaatgcagcaaggaatacctggagcgcttccggcgtgagggcaacgagaaagagatgcaaaggatcctgctgaactccgagcggctcaagtcccgcattgccgagatccatgagagccgcacgaagctggagcagcagctgcgggcccaggcctcggacaacagagagatcgacaagcgcatgaacagcctcaagccggacctcatgcagctgcgcaagatccgagaccagtacctcgtgtggctcacccagaaaggcgcccggcagaagaaaatcaacgagtggctggggattaaaaatgagactgaggaccagtacgcactcatggaggacgaggacgatctcccgcaccacgaggaacgcacttggtacgtgggcaagatcaaccgcacgcaggcagaggagatgctgagcggcaagcgggatggcaccttcctcatccgcgagagcagccagcggggctgctacgcctgctccgtggtagtggacggcgacaccaagcactgcgtcatctaccgcacggccaccggcttcggcttcgcggagccctacaacctgtacgggtcgctgaaggagctggtgctgcactaccagcacgcctcgctggtgcagcacaacgacgcgctcaccgtcaccctggcgcacccagtgcgcgccccgggccccggcccgccgcctgccgcccgctgagcaccgaggacccgccccaagcagagccgcccctgggcccgtctgcgccggaggctgcggcggcgggagccacggaccagaccagccacatccaggggtcctcatttctccggctctggctcttgtttggggttctctcaccctctttctctttccttccctcccccattctccagatctccctctgtctccttttctctgtctttcttggcccctgtctctctccatgttgggggtcctaactcccccaccccatatctacgtgtcctccgggcattgccctctccatggctctggtcaccctgaccctctgccctgcccaccgcaggtcccccggggtcccggaagccccttctggctgcacctgccatgtttacagagggcccctgggctgcgcggccccagcctgggcaccctgatttttaagccatagacctggggtcagggcaggaaggaacttcactctgctgcttccgagaacctcggccgtgacattcggggccgggcgggacccgccccacagactccaacttcccctccaaaccccgaagtgaaacccgccaccgggttacccccacaagggggccgctgcgagaagttcacccacccccgaaaaaataattaaactcgcaggccaggcacggtggctcatgcctgtaatcccagcactttgggaggccaagacgggcggatcttttgaggtcgggagttggaggccagcctggccaaaatggcaaaaccccgcatctactaaaatacaaaaattagccgggcgtggtggcggccgcctgtaatcccagctacttgggaggctgaggcgtgagaatctcttgaacccaggagatggaggttgcagtgagcagagatcgtgccactgcactccagcctgggtaacagagggagactcctccgtctcaaaaaaataaataaataaacttgtgagctggccccaacccctcctaggaatcacagctccccgtactggtgccgccgcagtggccaagttgcgacactgcccacggcccctccctctgatgcagattcagggcttctcttcgatcatgttgggttttgattctgtttttccttgactgcaaaaccctctttcctctcctcttttgggacaagagccctggttttctacgctgcccttggccaccacactgcctgccccacgagctgggaggcaggttttgtacggtacgttgttattgatatgatataaaacatcaaacgtcaaaaaaaaaaaaaaaaaaaaaaa

SEQ ID NO: 7 - protein, 728 aa, Homo sapiens
MAGPEGFQYRALYPFRRERPEDLELLPGDVLVVSRAALQALGVAEGGERCPQSVGWMPGLNERTRQRGDFPGTYVEFLGPVALARPGPRPRGPRPLPARPRDGAPEPGLTLPDLPEQFSPPDVAPPLLVKLVEAIERTGLDSESHYRPELPAPRTDWSLSDVDQWDTAALADGIKSFLLALPAPLVTPEASAEARRALREAAGPVGPALEPPTLPLHRALTLRFLLQHLGRVASRAPALGPAVRALGATFGPLLLRAPPPPSSPPPGGAPDGSEPSPDFPALLVEKLLQEHLEEQEVAPPALPPKPPKAKPASTVLANGGSPPSLQDAEWYWGDISREEVNEKLRDTPDGTFLVRDASSKIQGEYTLTLRKGGNNKLIKVFHRDGHYGFSEPLTFCSVVDLINHYRHESLAQYNAKLDTRLLYPVSKYQQDQIVKEDSVEAVGAQLKVYHQQYQDKSREYDQLYEEYTRTSQELQMKRTAIEAFNETIKIFEEQGQTQEKCSKEYLERFRREGNEKEMQRILLNSERLKSRIAEIHESRTKLEQQLRAQASDNREIDKRMNSLKPDLMQLRKIRDQYLVWLTQKGARQKKINEWLGIKNETEDQYALMEDEDDLPHHEERTWYVGKINRTQAEEMLSGKRDGTFLIRESSQRGCYACSVVVDGDTKHCVIYRTATGFGFAEPYNLYGSLKELVLHYQHASLVQHNDALTVTLAHPVRAPGPGPPPAAR

SEQ ID NO: 8 - DNA, 2427 bp, Homo sapiens
attacaaagaaagccggactcttttcttataactgagctcagccaaggaaactcttgcacaaatgtacaatactgtttggaatatggaagacctggatttagaatatgccaagacagatataaattgtggcacagacttgatgttttatatagaaatggacccaccagcactgcctcctaaaccaccaaaacctactactgtagccaacaacggtatgaataacaatatgtccttacaagatgctgaatggtactggggagatatctcgagggaagaagtgaatgaaaaacttcgagatacagcagacgggacctttttggtacgagatgcgtctactaaaatgcatggtgattatactcttacactaaggaaagggggaaataacaaattaatcaaaatatttcatcgagatgggaaatatggcttctctgacccattaaccttcagttctgtggttgaattaataaaccactaccggaatgaatctctagctcagtataatcccaaattggatgtgaaattactttatccagtatccaaataccaacaggatcaagttgtcaaagaagataatattgaagctgtagggaaaaaattacataaatataacactcagtttcaagaaaaaagtcgagaatatgatagattatatgaagaatatacccgcacatcccaggaaatccaaatgaaaaggacagctattgaagcatttaatgaaaccataaaaatatttgaagaacagtgccagacccaagagcggtacagcaaagaatacatagaaaagtttaaacgtgaaggcaatgagaaagaaatacaaaggattatgcataattatgataagttgaagtctcgaatcagtgaaattattgacagtagaagaagattggaagaagacttgaagaagcaggcagctgagtatcgagaaattgacaaacgtatgaacagcattaaaccagaccttatccagctgagaaagacgagagaccaatacttgatgtggttgactcaaaaaggtgttcggcaaaagaagttgaacgagtggttgggcaatgaaaacactgaagaccaatattcactggtggaagatgatgaagatttgccccatcatgatgagaagacatggaatgttggaagcagcaaccgaaacaaagctgaaaacctgttgcgagggaagcgagatggcacttttcttgtccgggagagcagtaaacagggctgctatgcctgctctgtagtggtggacggcgaagtaaagcattgtgtcataaacaaaacagcaactggctatggctttgccgagccctataacttgtacagctctctgaaagaactggtgctacattaccaacacacctcccttgtgcagcacaacgactccctcaatgtcacactagcctacccagtatatgcacagcagaggcgatgaagcgcttactctttgatccttctcctgaagttcagccaccctgaggcctctggaaagcaaagggctcctctccagtctgatctgtgaattgagctgcagaaacgaagccatctttctttggatgggactagagctttctttcacaaaaaagaagtaggggaagacatgcagcctaaggctgtatgatgaccacacgttcctaagctggagtgcttatcccttctttttctttttttctttggtttaatttaaagccacaaccacatacaacacaaagagaaaaagaaatgcaaaaatctctgcgtgcagggacaaagaggcctttaaccatggtgcttgttaatgctttctgaagctttaccagctgaaagttgggactctggagagcggaggagagagaggcagaagaaccctggcctgagaaggtttggtccagcctggtttagcctggatgttgctgtgcacggtggacccagacacatcgcactgtggattatttcattttgtaacaaatgaacgatatgtagcagaaaggcacgtccactcacaagggacgctttgggagaatgtcagttcatgtatgttcagaagaaattctgtcatagaaagtgccagaaagtgtttaacttgtcaaaaaacaaaaacccagcaacagaaaaatggagtttggaaaacaggacttaaaatgacattcagtatataaaatatgtacataatattggatgactaactatcaaatagatggatttgtatcaataccaaatagcttctgttttgttttgctgaaggctaaattcacagcgctatgcaattcttaattttcattaagttgttatttcagttttaaatgtaccttcagaataagcttccccaccccagtttttgttgcttgaagatattgttgtcccggatttttgttaatattcatttttgttatccttttttaaaagtaaatgtacaggatgccagcaaaaaaaaaaaaaaaaaaaaaaaaaaaaaa

SEQ ID NO: 9 - DNA, 1891 bp, Homo sapiens
ccgcttcggggaggaggacgctgaggaggcgccgagccgcgcagcgctgcgggggaggcgcccgcgccgacgcggggcccatggccaggaccaccagccagctgtatgacgccgtgcccatccagtccagcgtggtgttatgttcctgcccatccccatcaatggtgaggacccagactgagtccagcacgccccctggcattcctggtggcagcaggcagggccccgccatggacggcactgcagccgagcctcggcccggcgccggctccctgcagcatgcccagcctccgccgcagcctcggaagaagcggcctgaggacttcaagtttgggaaaatccttggggaaggctctttttccacggttgtcctggctcgagaactggcaacctccagagaatatgcgattaaaattctggagaagcgacatatcataaaagagaacaaggtcccctatgtaaccagagagcgggatgtcatgtcgcgcctggatcaccccttctttgttaagctttacttcacatttcaggacgacgagaagctgtatttcggccttagttatgccaaaaatggagaactacttaaatatattcgcaaaatcggttcattcgatgagacctgtacccgattttacacggctgagatcgtgtctgctttagagtacttgcacggcaagggcatcattcacagggaccttaaaccggaaaacattttgttaaatgaagatatgcacatccagatcacagattttggaacagcaaaagtcttatccccagagagcaaacaagccagggccaactcattcgtgggaacagcgcagtacgtttctccagagctgctcacggagaagtccgcctgtaagagttcagacctttgggctcttggatgcataatataccagcttgtggcaggactcccaccattccgagctggaaacgagtatcttatatttcagaagatcattaagttggaatatgactttccagaaaaattcttccctaaggcaagagacctcgtggagaaacttttggttttagatgccacaaagcggttaggctgtgaggaaatggaaggatacggacctcttaaagcacacccgttcttcgagtccgtcacgtgggagaacctgcaccagcagacgcctccgaagctcaccgcttacctgccggctatgtcggaagacgacgaggactgctatggcaattatgacaatctcctgagccagtttggctgcatgcaggtgtcttcgtcctcctcctcacactccctgtcagcctccgacacgggcctgccccagaggtcaggcagcaacatagagcagtacattcacgatctggactcgaactcctttgaactggacttacagttttccgaagatgagaagaggttgttgttggagaagcaggctggcggaaacccttggcaccagtttgtagaaaataatttaatactaaagatgggcccagtggataagcggaagggtttatttgcaagacgacgacagctgttgctcacagaaggaccacatttatattatgtggatcctgtcaacaaagttctgaaaggtgaaattccttggtcacaagaacttcgaccagaggccaagaattttaaaactttctttgtccacacgcctaacaggacgtattatctgatggaccccagcgggaacgcacacaagtggtgcaggaagatccaggaggtttggaggcagcgataccagagccacccggacgccgctgtgcagtgacgtggcctgcggccgggctgcccttcgctgccaggacacctgccccagcgcggcttggccgccatccgggacgcttccagaccacctgccagccatcacaaggggaacgcagaggcggaaaccttgcagcatttttattt

SEQ ID NO: 10 - protein, 556 aa, Homo sapiens
MARTTSQLYDAVPIQSSVVLCSCPSPSMVRTQTESSTPPGIPGGSRQGPAMDGTAAEPRPGAGSLQHAQPPPQPRKKRPEDFKFGKILGEGSFSTVVLARELATSREYAIKILEKRHIIKENKVPYVTRERDVMSRLDHPFFVKLYFTFQDDEKLYFGLSYAKNGELLKYIRKIGSFDETCTRFYTAEIVSALEYLHGKGIIHRDLKPENILLNEDMHIQITDFGTAKVLSPESKQARANSFVGTAQYVSPELLTEKSACKSSDLWALGCIIYQLVAGLPPFRAGNEYLIFQKIIKLEYDFPEKFFPKARDLVEKLLVLDATKRLGCEEMEGYGPLKAHPFFESVTWENLHQQTPPKLTAYLPAMSEDDEDCYGNYDNLLSQFGCMQVSSSSSSHSLSASDTGLPQRSGSNIEQYIHDLDSNSFELDLQFSEDEKRLLLEKQAGGNPWHQFVENNLILKMGPVDKRKGLFARRRQLLLTEGPHLYYVDPVNKVLKGEIPWSQELRPEAKNFKTFFVHTPNRTYYLMDPSGNAHKWCRKIQEVWRQRYQSHPDAAVQ

SEQ ID NO: 11 - DNA, 5238 bp, Homo sapiens
agtctggcaggcaggcaggcaatcggtccgagtggctgtcggctcttcagctctcccgctcggcgtcttccttcctcctcccggtcagcgtcggcggctgcaccggcggcggcgcagtccctgcgggaggggcgacaagagctgagcggcggccgccgagcgtcgagctcagcgcggcggaggcggcggcggcccggcagccaacatggcggcggcggcggcggcgggcgcgggcccggagatggtccgcgggcaggtgttcgacgtggggccgcgctacaccaacctctcgtacatcggcgagggcgcctacggcatggtgtgctctgcttatgataatgtcaacaaagttcgagtagctatcaagaaaatcagcccctttgagcaccagacctactgccagagaaccctgagggagataaaaatcttactgcgctccagacatgagaacatcattggaatcaatgacattattcgagcaccaaccatcgagcaaatgaaagatgtatatatagtacaggacctcatggaaacagatctttacaagctcttgaagacacaacacctcagcaatgaccatatctgctattttctctaccagatcctcagagggttaaaatatatccattcagctaacgttctgcaccgtgacctcaagccttccaacctgctgctcaacaccacctgtgatctcgagatctgtgactttggcctggcccgtgttgcagatccagaccatgatcacacagggttcctgacagaatatgtggccacacgttggtacagggctccagaaattatgttgaattccaagggctacaccaagtccattgatatttggtctgtaggctgcattctggcagaaatgctttctaacaggcccatctttccagggaagcattatcttgaccagctgaaccacattttgggtattcttggatccccatcacaagaagacctgaattgtataataaatttaaaagctaggaactatttgctttctcttccacacaaaaataaggtgccatggaacaggctgttcccaaatgctgactccaaagctctggacttattggacaaaatgttgacattcaacccacacaagaggattgaagtagaacaggctctggcccacccatatctggagcagtattacgacccgagtgacgagcccatcgccgaagcaccattcaagttcgacatggaattggatgacttgcctaaggaaaagctcaaagaactaatttttgaagagactgctagattccagccaggatacagatcttaaatttgtcaggacaagggctcagaggactggacgtgctcagacatcggtgttcttcttcccagttcttgacccctggtcctgtctccagcccgtcttggcttatccactttgactcctttgagccgtttggaggggcggtttctggtagttgtggcttttatgctttcaaagaatttcttcagtccagagaattcctcctggcagccctgtgtgtgtcacccattggtgacctgcggcagtatgtacttcagtgcacctactgcttactgttgctttagtcactaattgctttctggtttgaaagatgcagtggttcctccctctcctgaatccttttctacatgatgccctgctgaccatgcagccgcaccagagagagattcttccccaattggctctagtcactggcatctcactttatgatagggaaggctactacctagggcactttaagtcagtgacagccccttatttgcacttcaccttttgaccataactgtttccccagagcaggagcttgtggaaataccttggctgatgttgcagcctgcagcaagtgcttccgtctccggaatccttggggagcacttgtccacgtcttttctcatatcatggtagtcactaacatatataaggtatgtgctattggcccagcttttagaaaatgcagtcatttttctaaataaaaaggaagtactgcacccagcagtgtcactctgtagttactgtggtcacttgtaccatatagaggtgtaacacttgtcaagaagcgttatgtgcagtacttaatgtttgtaagacttacaaaaaaagatttaaagtggcagcttcactcgacatttggtgagagaagtacaaaggttgcagtgctgagctgtgggcggtttctggggatgtcccagggtggaactccacatgctggtgcatatacgcccttgagctacttcaaatgtgggtgtttcagtaaccacgttccatgcctgaggatttagcagagaggaacactgcgtctttaaatgagaaagtatacaattctttttccttctacagcatgtcagcatctcaagttcatttttcaacctacagtataacaatttgtaataaagcctccaggagctcatgacgtgaagcactgttctgtcctcaagtactcaaatatttctgatactgctgagtcagactgtcagaaaaagctagcactaactcgtgtttggagctctatccatattttactgatctctttaagtatttgttcctgccactgtgtactgtggagttgactcggtgttctgtcccagtgcggtgcctcctcttgacttccccactgctctctgtggtgagaaatttgccttgttcaataattactgtaccctcgcatgactgttacagctttctgtgcagagatgactgtccaagtgccacatgcctacgattgaaatgaaaactctattgttacctctgagttgtgttccacggaaaatgctatccagcagatcatttaggaaaaataattctatttttagcttttcatttctcagctgtccttttttcttgtttgatttttgacagcaatggagaatgggttatataaagactgcctgctaatatgaacagaaatgcatttgtaattcatgaaaataaatgtacatcttctatcttcacattcatgttaagattcagtgttgctttcctctggatcagcgtgtctgaatggacagtcaggttcaggttgtgctgaacacagaaatgctcacaggcctcactttgccgcccaggcactggcccagcacttggatttacataagatgagttagaaaggtacttctgtagggtcctttttacctctgctcggcagagaatcgatgctgtcatgttcctttattcacaatcttaggtctcaaatattctgtcaaaccctaacaaagaagccccgacatctcaggttggattccctggttctctataaagagggcctgcccttgtgccccagaggtgctgctgggcacagccaagagttgggaagggccgccccacagtacgcagtcctcaccacccagcccagggtgctcacgctcaccactcctgtggctgaggaaggatagctggctcatcctcggaaaacagacccacatctctattcttgccctggaatacgcgcttttcacttgcgtgctcagagctgccgtctgaaggtccacacagcattgacgggacacagaaatgtgactgttaccggataacactgattagtcagttttcatttataaaaaagcattgacagttttattactcttgtttctttttaaatggaaagttactattataaggttaatttggagtcctcttctaaatagaaaaccatatccttggctactaacatctggagactgtgagctccttcccattccccttcctggtactgtggagtcagattggcatgaaaccactaacttcattctagaatcattgtagccataagttgtgtgctttttattaatcatgccaaacataatgtaactgggcagagaatggtcctaaccaaggtacctatgaaaagcgctagctatcatgtgtagtagatgcatcattttggctcttcttacatttgtaaaaatgtacagattaggtcatcttaattcatattagtgacacggaacagcacctccactatttgtatgttcaaataagctttcagactaatagcttttttggtgtctaaaatgtaagcaaaaaattcctgctgaaacattccagtcctttcatttagtataaaagaaatactgaacaagccagtgggatggaattgaaagaactaatcatgaggactctgtcctgacacaggtcctcaaagctagcagagatacgcagacattgtggcatctgggtagaagaatactgtattgtgtgtgcagtgcacagtgtgtggtgtgtgcacactcattccttctgctcttgggcacaggcagtgggtgtagaggtaaccagtagctttgagaagctacatgtagctcaccagtggttttctctaaggaatcacaaaagtaaactacccaaccacatgccacgtaatatttcagccattcagaggaaactgttttctctttatttgcttatatgttaatatggtttttaaattggtaacttttatatagtatggtagcagtatgttaatacacacatacatacgcacacatgctttgggtccttccataatacttttatatttgtaaatcaatgttttggagcaatcccaagtttaagggaaatatttttgtaaatgtaatggttttgaaaatctgagcaatccttttgcttatacatttttaaagcatttgtgctttaaaattgttatgctggtgtttgaaacatgatactcctgtggtgcagatgagaagctataacagtgaatatgtggtttctcttacgtcatccaccttgacatgatgggtcagaaacaaatggaaatccagagcaagtcctccagggttgcaccaggtttacctaaagcttgttgccttttcttgtgctgtttatgcgtgtagagcactcaagaaagttctgaaactgctttgtatctgctttgtactgttggtgccttcttggtattgtaccccaaaattctgcatagattatttagtataatggtaagttaaaaaatgttaaaggaagattttattaagaatctgaatgtttattcattatattgttacaatttaacattaacatttatttgtggtatttgtgatttggttaatctgtataaaaattgtaagtagaaaggtttatatttcatcttaattcttttgatgttgtaaacgtactttttaaaagatggattatttgaatgtttatggcacctgacttgtaaaaaaaaaaaactacaaaaaaatccttagaatcattaaattgtgtccctgtattaccaaaataacacaaaaaaaaaaaaaaaa

SEQ ID NO: 12 - protein, 360 aa, Homo sapiens [sequence omitted]

SEQ ID NO: 13 - DNA, 1863 bp, Homo sapiens
gcagactcagttcctggagaaagatggcgacagccgagaagcagaaacacgacgggcgggtgaagatcggccactacattctgggtgacacgctgggggtcggcaccttcggcaaagtgaaggttggcaaacatgaattgactgggcataaagtagctgtgaagatactcaatcgacagaagattcggagccttgatgtggtaggaaaaatccgcagagaaattcagaacctcaagcttttcaggcatcctcatataattaaactgtaccaggtcatcagtacaccatctgatattttcatggtgatggaatatgtctcaggaggagagctatttgattatatctgtaagaatggaaggctggatgaaaaagaaagtcggcgtctgttccaacagatcctttctggtgtggattattgtcacaggcatatggtggtccatagagatttgaaacctgaaaatgtcctgcttgatgcacacatgaatgcaaagatagctgattttggtctttcaaacatgatgtcagatggtgaatttttaagaacaagttgtggctcacccaactatgctgcaccagaagtaatttcaggaagattgtatgcaggcccagaggtagatatatggagcagtggggttattctctatgctttattatgtggaacccttccatttgatgatgaccatgtgccaactctttttaagaagatatgtgatgggatcttctatacccctcaatatttaaatccttctgtgattagccttttgaaacatatgctgcaggtggatcccatgaagagggcctcaatcaaagatatcagggaacatgaatggtttaaacaggaccttccaaaatatctctttcctgaggatccatcatatagttcaaccatgattgatgatgaagccttaaaagaagtatgtgaaaagtttgagtgctcagaagaggaagttctcagctgtctttacaacagaaatcaccaggatcctttggcagttgcctaccatctcataatagataacaggagaataatgaatgaagccaaagatttctatttggcgacaagcccacctgattcttttcttgatgatcatcacctgactcggccccatcctgaaagagtaccattcttggttgctgaaacaccaagggcacgccatacccttgatgaattaaatccacagaaatccaaacaccaaggtgtaaggaaagcaaaatggcatttaggaattagaagtcaaagtcgaccaaatgatattatggcagaagtatgtagagcaatcaaacaattggattatgaatggaaggttgtaaacccatattatttgcgtgtacgaaggaagaatcctgtgacaagcacttactccaaaatgagtctacagttataccaagtggatagtagaacttatctactggatttccgtagtattgatgatgaaattacagaagccaaatcagggactgctactccacagagatcgggatcagttagcaactatcgatcttgccaaaggagtgattcagatgctgaggctcaaggaaaatcctcagaagtttctcttacctcatctgtgacctcacttgactcttctcctgttgacctaactccaagacctggaagtcacacaatagaattttttgagatgtgtgcaaatctaattaaaattcttgcacaataaacagaaaactttgcttatttcttttgcagcaataagcatgcataataagtcacagccaaatgcttccatttgtaatcaagttatacataattataaccgagggctggcgttttggaatcgaatttcgacagggattggaacatgatttatagttaaaagcctaatatcgagaaatgaattaagatca

SEQ ID NO: 14 - protein, 559 aa, Homo sapiens
MRRLSSWRKMATAEKQKHDGRVKIGHYILGDTLGVGTFGKVKVGKHELTGHKVAVKILNRQKIRSLDVVGKIRREIQNLKLFRHPHIIKLYQVISTPSDIFMVMEYVSGGELFDYICKNGRLDEKESRRLFQQILSGVDYCHRHMVVHRDLKPENVLLDAHMNAKIADFGLSNMMSDGEFLRTSCGSPNYAAPEVISGRLYAGPEVDIWSSGVILYALLCGTLPFDDDHVPTLFKKICDGIFYTPQYLNPSVISLLKHMLQVDPMKRATIKDIREHEWFKQDLPKYLFPEDPSYSSTMIDDEALKEVCEKFECSEEEVLSCLYNRNHQDPLAVAYHLIIDNRRIMNEAKDFYLATSPPDSFLDDHHLTRPHPERVPFLVAETPRARHTLDELNPQKSKHQGVRKAKWHLGIRSQSRPNDIMAEVCRAIKQLDYEWKVVNPYYLRVRRKNPVTSTYSKMSLQLYQVDSRTYLLDFRSIDDEITEAKSGTATPQRSGSVSNYRSCQRSDSDAEAQGKSSEVSLTSSVTSLDSSPVDLTPRPGSHTIEFFEMCANLIKILAQ

SEQ ID NO: 15 - DNA, 3919 bp, Homo sapiens
ctttccctctcccaacatggcggcctcagcaaaaaagaagaataagaaggggaagactatctccctaacagactttctggctgaggatgggggtactggtggaggaagcacctatgtttccaaaccagtcagctgggctgatgaaacggatgacctggaaggagatgtttcgaccacttggcacagtaacgatgacgatgtgtatagggcgcctccaattgaccgttccatccttcccactgctccacgggctgctcgggaacccaatatcgaccggagccgtcttcccaaatcgccaccctacactgcttttctaggaaacctaccctatgatgttacagaagagtcaattaaggaattctttcgaggattaaatatcagtgcagtgcgtttaccacgtgaacccagcaatccagagaggttgaaaggttttggttatgctgaatttgaggacctggattccctgctcagtgccctgagtctcaatgaagagtctctaggtaacaggagaattcgagtggacgttgctgatcaagcacaggataaagacagggatgatcgttcttttggccgtgatagaaatcgggattctgacaaaacagatacagactggagggctcgtcctgctacagacagctttgatgactacccacctagaagaggtgatgatagctttggagacaagtatcgagatcgttatgattcagaccggtatcgggatgggtatcgggatgggtatcgggatggcccacgccgggatatggatcgatatggtggccgggatcgctatgatgaccgaggcagcagagactatgatagaggctatgattcccggataggcagtggcagaagagcatttggcagtgggtatcgcagggatgatgactacagaggaggcggggaccgctatgaagaccgatatgacagacgggatgatcggtcgtggagctccagagatgattactctcgggatgattataggcgtgatgatagaggtcccccccaaagacccaaactgaatctaaagcctcggagtactcctaaggaagatgattcctctgctagtacctcccagtccactcgagctgcttctatctttggaggggcaaagcctgttgacacagctgctagagaaagagaagtagaagaacggctacagaaggaacaagagaagttgcagcgtcagctggatgagccaaaactagaacgacggcctcgggagagacacccaagctggcgaagtgaagaaactcaggaacgggaacggtcgaggacaggaagtgagtcatcacaaactgggacctccaccacatctagcagaaatgcacgaaggagagagagtgagaagtctctagaaaatgaaacactcaataaggaggaagattgccactctccaacttctaaacctcccaaacctgatcagcccctaaaggtaatgccagcccctccaccaaaggagaatgcttgggtgaagcgaagttctaaccctcctgctcgatctcagagctcagacacagagcagcagtcccctacaagtggtgggggaaaagtagctccagctcaaccatctgaggaaggaccaggaaggaaagatgaaaataaagtagatgggatgaatgccccaaaaggccaaactgggaactctagccgtggtccaggagacggagggaacagagaccactggaaggagtcagataggaaagatggcaaaaaggatcaagactccagatctgcacctgagccaaagaaacctgaggaaaatccagcttctaagttcagttctgcaagcaagtatgctgctctctctgttgatggtgaagatgaaaatgagggagaagattatgccgaatagacctctacatcctgtgcttttctcctagtttctctccaccctggaacattcgagagcaaatcaaaacctctatccagacaagacaaaataaaactcaacatctcctgaagacctttcttacctttttttaaaaacaaaaaatgaaattattttgcatgctgctgcagcctttaaagtattgaagtaactggagaattgccaatacagccagagagaaagggactacagctttttagaggaaaagttgtggtgcgttatgtcaccatgcagttgccagtgtgattagtgcctaggggtctccatttagcagaaatggtaatgacagtgatataatgcctggaacctggttgggcagtaggggagggaggtagaaggaaaagtgtgagatttctaccttttagtttttatcctattgtggcatatatgaattctcaaacattatctgaataaattttccactcttggaaaggtagatttagcctcaagttgttctagtctccaggaggctgccagcccctcctcttatttaattctgagttttgggggccagcctagagggaatccctttttttttttaaccccccaggggggtagttgggagtgagactataggccataaagaatgggactgcattggaccaaaataaatgggaaaatcgtggtttgaaaagaagcttttgggaagtgatgagtcattttgcaccaggtaataggggaaaattgtgtgacctccagcaaacacatgaatggttatttcctggagccggaagcacttgggggtcgtggtaattcccagtgttttctgtgtcctagttttaccctttctaaacactgtcctttttgaaagttttgaatatatccacattctattgaaaccttgaaactaaaaatttagactcttatcgtcatcttaagttcttcatggtactcttaacctcccaaaaagcagtatctaagtcacatacatgatgtcttgggcattttctgagccatggagaactctgaaaggaagaatcgctgcttttctcaagcaaatcggtttcttgatgtcttttggttctccttgcctgctcctgatgcttggaccccttttattgatcagagtgctctagaataatggatggtcttggatgatggataaatagggacagggacagttaaattgggagcctttcttacaaccttgatgggatttttccccccaagtttccttctccactgaaatgccacactaatgcttgttggattcatgaggtggccagaccaatgtgttgttttgttgttgttttttttttttaagcttcccttgagagaataaatggtaatggagagaactatttaacaaggtcctggtttctcttgcaacacagtagctaaacttgcctgcttttatatgcatttttgtagggatcagcttggtagacagtattagcggagaaacaccttgatcttggtttgcaagcccttctcccatcagtcctagattaggccctgttcagccatgcaggggtgttggtttatgcgtgctgcagcagtgggcataatgaatataatttacccagtggacaaaggtgtgtaccaagtgaatttaaataattggtgtggattggccagtagctaagtgggcttttaaagagtattgaagattgaaagggtttttctttcttttttaaaaaagaaaaacaaactattgattgtagataatgaaaagctagggtttgccctcttcatgtctactctccttccaaatagttatatccaaaactgtttttccctctcccctaccttgtcccccctattaaaatagaaacagggattgattaatgtcccgctcctgaatacatgtaaaatttgtacaaaaatatcttctatgaaaatgatttgtaatctgtagacttattacctgggagatgtcttgatgtaaaatcccatcctttgggttgtgggttttttgttttctccaaataaatctgatctttaaagttcaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 16 - protein, 611 aa, Homo sapiens
MAASAKKKNKKGKTISLTDFLAEDGGTGGGSTYVSKPVSWADETDDLEGDVSTTWHSNDDDVYRAPPIDRSILPTAPRAAREPNIDRSRLPKSPPYTAFLGNLPYDVTEESIKEFFRGLNISAVRLPREPSNPERLKGFGYAEFEDLDSLLSALSLNEESLGNRRIRVDVADQAQDKDRDDRSFGRDRNRDSDKTDTDWRARPATDSFDDYPPRRGDDSFGDKYRDRYDSDRYRDGYRDGYRDGPRRDMDRYGGRDRYDDRGSRDYDRGYDSRIGSGRRAFGSGYRRDDDYRGGGDRYEDRYDRRDDRSWSSRDDYSRDDYRRDDRGPPQRPKLNLKPRSTPKEDDSSASTSQSTRAASIFGGAKPVDTAAREREVEERLQKEQEKLQRQLDEPKLERRPRERHPSWRSEETQERERSRTGSESSQTGTSTTSSRNARRRESEKSLENETLNKEEDCHSPTSKPPKPDQPLKVMPAPPPKENAWVKRSSNPPARSQSSDTEQQSPTSGGGKVAPAQPSEEGPGRKDENKVDGMNAPKGQTGNSSRGPGDGGNRDHWKESDRKDGKKDQDSRSAPEPKKPEENPASKFSSASKYAALSVDGEDENEGEDYAE

SEQ ID NO: 17 - DNA, 877 bp, Homo sapiens
ggggcgaggcggagcgaggctggaggcgcgggagggcagcgagaggttcgcgggtgcagcgcacaggagaccatgtccgggggcagcagctgcagccagaccccaagccgggccatccccgccactcgccgggtggtgctcggcgacggcgtgcagctcccgcccggggactacagcacgacccccggcggcacgctcttcagcaccaccccgggaggtaccaggatcatctatgaccggaaattcctgatggagtgtcggaactcacctgtgaccaaaacacccccaagggatctgcccaccattccgggggtcaccagcccttccagtgatgagccccccatggaagccagccagagccacctgcgcaatagcccagaagataagcgggcgggcggtgaagagtcacagtttgagatggacatttaaagcaccagccatcgtgtggagcactaccaaggggcccctcagggccttcctgggaggagtcccaccagccaggccttatgaaagtgatcatactgggcaggcgttggcgtggggtcggacaccccagccctttctccctcactcagggcacctgccccctcctcttcgtgaacaccagcagatacctccttgtgcctccactgatgcaggagctgccaccccaaggggagtgacccctgccagcacaccctgcagccaagggccaggaagtggacaagaacgaacccttccttccgaatgatcagcagttccagcccctcgctgctgggggcgcaaccaccccttccttaggttgatgtgcttgggaaagctccctccccctccttccccaagagaggaaataaaagccaccttcgccctagggccaagaaaaaaaaaaaaaaaaaaa

SEQ ID NO: 18 - protein, 118 aa, Homo sapiens
MSGGSSCSQTPSRAIPATRRVVLGDGVQLPPGDYSTTPGGTLFSTTPGGTRIIYDRKFLMECRNSPVTKTPPRDLPTIPGVTSPSSDEPPMEASQSHLRNSPEDKRAGGEESQFEMDI

SEQ ID NO: 19 - DNA, 8733 bp, Homo sapiens
gctcccggcttagaggacagcggggaaggcgggcggtggggcagggggcctgaagcggcggtaccggtgctggcggcggcagctgaggccttggccgaagccgcgcgaacctcagggcaagatgcttggaaccggacctgccgccgccaccaccgctgccaccacatctagcaatgtgagcgtcctgcagcagtttgccagtggcctaaagagccggaatgaggaaaccagggccaaagccgccaaggagctccagcactatgtcaccatggaactccgagagatgagtcaagaggagtctactcgcttctatgaccaactgaaccatcacatttttgaattggtttccagctcagatgccaatgagaggaaaggtggcatcttggccatagctagcctcataggagtggaaggtgggaatgccacccgaattggcagatttgccaactatcttcggaacctcctcccctccaatgacccagttgtcatggaaatggcatccaaggccattggccgtcttgccatggcaggggacacttttaccgctgagtacgtggaatttgaggtgaagcgagccctggaatggctgggtgctgaccgcaatgagggccggagacatgcagctgtcctggttctccgtgagctggccatcagcgtccctaccttcttcttccagcaagtgcaacccttctttgacaacatttttgtggccgtgtgggaccccaaacaggccatccgtgagggagctgtagccgcccttcgtgcctgtctgattctcacaacccagcgtgagccgaaggagatgcagaagcctcagtggtacaggcacacatttgaagaagcagagaagggatttgatgagaccttggccaaagagaagggcatgaatcgggatgatcggatccatggagccttgttgatccttaacgagctggtccgaatcagcagcatggagggagagcgtctgagagaagaaatggaagaaatcacacagcagcagctggtacacgacaagtactgcaaagatctcatgggcttcggaacaaaacctcgtcacattacccccttcaccagtttccaggctgtacagccccagcagtcaaatgccttggtggggctgctggggtacagctctcaccaaggcctcatgggatttgggacctcccccagtccagctaagtccaccctggtggagagccggtgttgcagagacttgatggaggagaaatttgatcaggtgtgccagtgggtgctgaaatgcaggaatagcaagaactcgctgatccaaatgacaatccttaatttgttgccccgcttggctgcattccgaccttctgccttcacagatacccagtatctccaagataccatgaaccatgtcctaagctgtgtcaagaaggagaaggaacgtacagcggccttccaagccctggggctactttctgtggctgtgaggtctgagtttaaggtctatttgcctcgcgtgctggacatcatccgagcggccctgcccccaaaggacttcgcccataagaggcagaaggcaatgcaggtggatgccacagtcttcacttgcatcagcatgctggctcgagcaatggggccaggcatccagcaggatatcaaggagctgctggagcccatgctggcagtgggactaagccctgccctcactgcagtgctctacgacctgagccgtcagattccacagctaaagaaggacattcaagatgggctactgaaaatgctgtccctggtccttatgcacaaaccccttcgccacccaggcatgcccaagggcctggcccatcagctggcctctcctggcctcacgaccctccctgaggccagcgatgtgggcagcatcactcttgccctccgaacgcttggcagctttgaatttgaaggccactctctgacccaatttgttcgccactgtgcggatcatttcctgaacagtgagcacaaggagatccgcatggaggctgcccgcacctgctcccgcctgctcacaccctccatccacctcatcagtggccatgctcatgtggttagccagaccgcagtgcaagtggtggcagatgtgcttagcaaactgctcgtagttgggataacagatcctgaccctgacattcgctactgtgtcttggcgtccctggacgagcgctttgatgcacacctggcccaggcggagaacttgcaggccttgtttgtggctctgaatgaccaggtgtttgagatccgggagctggccatctgcactgtgggccgactcagtagcatgaaccctgcctttgtcatgcctttcctgcgcaagatgctcatccagattttgacagagttggagcacagtgggattggaagaatcaaagagcagagtgcccgcatgctggggcacctggtctccaatgccccccgactcatccgcccctacatggagcctattctgaaggcattaattttgaaactgaaagatccagaccctgatccaaacccaggtgtgatcaataatgtcctggcaacaataggagaattggcacaggttagtggcctggaaatgaggaaatgggttgatgaactttttattatcatcatggacatgctccaggattcctctttgttggccaaaaggcaggtggctctgtggaccctgggacagttggtggccagcactggctatgtagtagagccctacaggaagtaccctactttgcttgaggtgctactgaattttctgaagactgagcagaaccagggtacacgcagagaggccatccgtgtgttagggcttttaggggctttggatccttacaagcacaaagtgaacattggcatgatagaccagtcccgggatgcctctgctgtcagcctgtcagaatccaagtcaagtcaggattcctctgactatagcactagtgaaatgctggtcaacatgggaaacttgcctctggatgagttctacccagctgtgtccatggtggccctgatgcggatcttccgagaccagtcactctctcatcatcacaccatggttgtccaggccatcaccttcatcttcaagtccctgggactcaaatgtgtgcagttcctgccccaggtcatgcccacgttccttaacgtcattcgagtctgtgatggggccatccgggaatttttgttccagcagctgggaatgttggtgtcctttgtgaagagccacatcagaccttatatggatgaaatagtcaccctcatgagagaattctgggtcatgaacacctcaattcagagcacgatcattcttctcattgagcaaattgtggtagctcttgggggtgaatttaagctctacctgccccagctgatcccacacatgctgcgtgtcttcatgcatgacaacagcccaggccgcattgtctctatcaagttactggctgcaatccagctgtttggcgccaacctggatgactacctgcatttactgctgcctcctattgttaagttgtttgatgcccctgaagctccactgccatctcgaaaggcagcgctagagactgtggaccgcctgacggagtccctggatttcactgactatgcctcccggatcattcaccctattgttcgaacactggaccagagcccagaactgcgctccacagccatggacacgctgtcttcacttgtttttcagctggggaagaagtaccaaattttcattccaatggtgaataaagttctggtgcgacaccgaatcaatcatcagcgctatgatgtgctcatctgcagaattgtcaagggatacacacttgctgatgaagaggaggatcctttgatttaccagcatcggatgcttaggagtggccaaggggatgcattggctagtggaccagtggaaacaggacccatgaagaaactgcacgtcagcaccatcaacctccaaaaggcctggggcgctgccaggagggtctccaaagatgactggctggaatggctgagacggctgagcctggagctgctgaaggactcatcatcgccctccctgcgctcctgctgggccctggcacaggcctacaacccgatggccagggatctcttcaatgctgcatttgtgtcctgctggtctgaactgaatgaagatcaacaggatgagctcatcagaagcatcgagttggccctcacctcacaagacatcgctgaagtcacacagaccctcttaaacttggctgaattcatggaacacagtgacaagggccccctgccactgagagatgacaatggcattgttctgctgggtgagagagctgccaagtgccgagcatatgccaaagcactacactacaaagaactggagttccagaaaggccccacccctgccattctagaatctctcatcagcattaataataagctacagcagccggaggcagcggccggagtgttagaatatgccatgaaacactttggagagctggagatccaggctacctggtatgagaaactgcacgagtgggaggatgcccttgtggcctatgacaagaaaatggacaccaacaaggacgacccagagctgatgctgggccgcatgcgctgcctcgaggccttgggggaatggggtcaactccaccagcagtgctgtgaaaagtggaccctggttaatgatgagacccaagccaagatggcccggatggctgctgcagctgcatggggtttaggtcagtgggacagcatggaagaatacacctgtatgatccctcgggacacccatgatggggcattttatagagctgtgctggcactgcatcaggacctcttctccttggcacaacagtgcattgacaaggccagggacctgctggatgctgaattaactgcgatggcaggagagagttacagtcgggcatatggggccatggtttcttgccacatgctgtccgagctggaggaggttatccagtacaaacttgtccccgagcgacgagagatcatccgccagatctggtgggagagactgcagggctgccagcgtatcgtagaggactggcagaaaatccttatggtgcggtcccttgtggtcagccctcatgaagacatgagaacctggctcaagtatgcaagcctgtgcggcaagagtggcaggctggctcttgctcataaaactttagtgttgctcctgggagttgatccgtctcggcaacttgaccatcctctgccaacagttcaccctcaggtgacctatgcctacatgaaaaacatgtggaagagtgcccgcaagatcgatgccttccagcacatgcagcattttgtccagaccatgcagcaacaggcccagcatgccatcgctactgaggaccagcagcataagcaggaactgcacaagctcatggcccgatgcttcctgaaacttggagagtggcagctgaatctacagggcatcaatgagagcacaatccccaaagtgctgcagtactacagcgccgccacagagcacgaccgcagctggtacaaggcctggcatgcgtgggcagtgatgaacttcgaagctgtgctacactacaaacatcagaaccaagcccgcgatgagaagaagaaactgcgtcatgccagcggggccaacatcaccaacgccaccactgccgccaccacggccgccactgccaccaccactgccagcaccgagggcagcaacagtgagagcgaggccgagagcaccgagaacagccccaccccatcgccgctgcagaagaaggtcactgaggatctgtccaaaaccctcctgatgtacacggtgcctgccgtccagggcttcttccgttccatctccttgtcacgaggcaacaacctccaggatacactcagagttctcaccttatggtttgattatggtcactggccagatgtcaatgaggccttagtggagggggtgaaagccatccagattgatacctggctacaggttatacctcagctcattgcaagaattgatacgcccagacccttggtgggacgtctcattcaccagcttctcacagacattggtcggtaccacccccaggccctcatctacccactgacagtggcttctaagtctaccacgacagcccggcacaatgcagccaacaagattctgaagaacatgtgtgagcacagcaacaccctggtccagcaggccatgatggtgagcgaggagctgatccgagtggccatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcacccaagcctgggacctctattatcatgtgttccgacgaatctcaaagcagctgcctcagctcacatccttagagctgcaatatgtttccccaaaacttctgatgtgccgggaccttgaattggctgtgccaggaacatatgaccccaaccagccaatcattcgcattcagtccatagcaccgtctttgcaagtcatcacatccaagcagaggccccggaaattgacacttatgggcagcaacggacatgagtttgttttccttctaaaaggccatgaagatctgcgccaggatgagcgtgtgatgcagctcttcggcctggttaacacccttctggccaatgacccaacatctcttcggaaaaacctcagcatccagagatacgctgtcatccctttatcgaccaactcgggcctcattggctgggttccccactgtgacacactgcacgccctcatccgggactacagggagaagaagaagatccttctcaacatcgagcatcgcatcatgttgcggatggctccggactatgaccacttgactctgatgcagaaggtggaggtgtttgagcatgccgtcaataatacagctggggacgacctggccaagctgctgtggctgaaaagccccagctccgaggtgtggtttgaccgaagaaccaattatacccgttctttagcggtcatgtcaatggttgggtatattttaggcctgggagatagacacccatccaacctgatgctggaccgtctgagtgggaagatcctgcacattgactttggggactgctttgaggttgctatgacccgagagaagtttccagagaagattccatttagactaacaagaatgttgaccaatgctatggaggttacaggcctggatggcaactacagaatcacatgccacacagtgatggaggtgctgcgagagcacaaggacagtgtcatggccgtgctggaagcctttgtctatgaccccttgctgaactggaggctgatggacacaaataccaaaggcaacaagcgatcccgaacgaggacggattcctactctgctggccagtcagtcgaaattttggacggtgtggaacttggagagccagcccataagaaaacggggaccacagtgccagaatctattcattctttcattggagacggtttggtgaaaccagaggccctaaataagaaagctatccagattattaacagggttcgagataagctcactggtcgggacttctctcatgatgacactttggatgttccaacgcaagttgagctgctcatcaaacaagcgacatcccatgaaaacctctgccagtgctatattggctggtgccctttctggtaactggaggcccagatgtgcccatcacgttttttctgaggcttttgtactttagtaaatgcttccactaaactgaaaccatggtgagaaagtttgactttgttaaatattttgaaatgtaaatgaaaagaactactgtatattaaaagttggtttgaaccaactttctagctgctgttgaagaatatattgtcagaaacacaaggcttgatttggttcccaggacagtgaaacatagtaataccacgtaaatcaagccattcattttggggaacagaagatccataactttagaaatacgggttttgacttaactcacaagagaactcatcataagtacttgctgatggaagaatgacctagttgctcctctcaacatgggtacagcaaactcagcacagccaagaagcctcaggtcgtggagaacatggattaggatcctagactgtaaagacacagaagatgctgacctcacccctgccacctatcccaagacctcactggtctgtggacagcagcagaaatgtttgcaagataggccaaaatgagtacaaaaggtctgtcttccatcagacccagtgatgctgcgactcacacgcttcaattcaagacctgaccgctagtagggaggtttattcagatcgctggcagcctcggctgagcagatgcacagaggggatcactgtgcagtgggaccaccctcactggccttctgcagcagggttctgggatgttttcagtggtcaaaatactctgtttagagcaagggctcagaaaacagaaatactgtcatggaggtgctgaacacagggaaggtctggtacatattggaaattatgagcagaacaaatactcaactaaatgcacaaagtataaagtgtagccatgtctagacaccatgttgtatcagaataatttttgtgccaataaatgacatcagaattttaaacatatgtaaaaaaaaa

SEQ ID NO: 20 - protein, 2549 aa, Homo sapiens
MLGTGPAAATTAATTSSNVSVLQQFASGLKSRNEETRAKAAKELQHYVTMELREMSQEESTRFYDQLNHHIFELVSSSDANERKGGILAIASLIGVEGGNATRIGRFANYLRNLLPSNDPVVMEMASKAIGRLAMAGDTFTAEYVEFEVKRALEWLGADRNEGRRHAAVLVLRELAISVPTFFFQQVQPFFDNIFVAVWDPKQAIREGAVAALRACLILTTQREPKEMQKPQWYRHTFEEAEKGFDETLAKEKGMNRDDRIHGALLILNELVRISSMEGERLREEMEEITQQQLVHDKYCKDLMGFGTKPRHITPFTSFQAVQPQQSNALVGLLGYSSHQGLMGFGTSPSPAKSTLVESRCCRDLMEEKFDQVCQWVLKCRNSKNSLIQMTILNLLPRLAAFRPSAFTDTQYLQDTMNHVLSCVKKEKERTAAFQALGLLSVAVRSEFKVYLPRVLDIIRAALPPKDFAHKRQKAMQVDATVFTCISMLARAMGPGIQQDIKELLEPMLAVGLSPALTAVLYDLSRQIPQLKKDIQDGLLKMLSLVLMHKPLRHPGMPKGLAHQLASPGLTTLPEASDVGSITLALRTLGSFEFEGHSLTQFVRHCADHFLNSEHKEIRMEAARTCSRLLTPSIHLISGHAHVVSQTAVQVVADVLSKLLVVGITDPDPDIRYCVLASLDERFDAHLAQAENLQALFVALNDQVFEIRELAICTVGRLSSMNPAFVMPFLRKMLIQILTELEHSGIGRIKEQSARMLGHLVSNAPRLIRPYMEPILKALILKLKDPDPDPNPGVINNVLATIGELAQVSGLEMRKWVDELFIIIMDMLQDSSLLAKRQVALWTLGQLVASTGYVVEPYRKYPTLLEVLLNFLKTEQNQGTRREAIRVLGLLGALDPYKHKVNIGMIDQSRDASAVSLSESKSSQDSSDYSTSEMLVNMGNLPLDEFYPAVSMVALMRIFRDQSLSHHHTMVVQAITFIFKSLGLKCVQFLPQVMPTFLNVIRVCDGAIREFLFQQLGMLVSFVKSHIRPYMDEIVTLMREFWVMNTSIQSTIILLIEQIVVALGGEFKLYLPQLIPHMLRVFMHDNSPGRIVSIKLLAAIQLFGANLDDYLHLLLPPIVKLFDAPEAPLPSRKAALETVDRLTESLDFTDYASRIIHPIVRTLDQSPELRSTAMDTLSSLVFQLGKKYQIFIPMVNKVLVRHRINHQRYDVLICRIVKGYTLADEEEDPLIYQHRMLRSGQGDALASGPVETGPMKKLHVSTINLQKAWGAARRVSKDDWLEWLRRLSLELLKDSSSPSLRSCWALAQAYNPMARDLFNAAFVSCWSELNEDQQDELIRSIELALTSQDIAEVTQTLLNLAEFMEHSDKGPLPLRDDNGIVLLGERAAKCRAYAKALHYKELEFQKGPTPAILESLISINNKLQQPEAAAGVLEYAMKHFGELEIQATWYEKLHEWEDALVAYDKKMDTNKDDPELMLGRMRCLEALGEWGQLHQQCCEKWTLVNDETQAKMARMAAAAAWGLGQWDSMEEYTCMIPRDTHDGAFYRAVLALHQDLFSLAQQCIDKARDLLDAELTAMAGESYSRAYGAMVSCHMLSELEEVIQYKLVPERREIIRQIWWERLQGCQRIVEDWQKILMVRSLVVSPHEDMRTWLKYASLCGKSGRLALAHKTLVLLLGVDPSRQLDHPLPTVHPQVTYAYMKNMWKSARKIDAFQHMQHFVQTMQQQAQHAIATEDQQHKQELHKLMARCFLKLGEWQLNLQGINESTIPKVLQYYSAATEHDRSWYKAWHAWAVMNFEAVLHYKHQNQARDEKKKLRHASGANITNATTAATTAATATTTASTEGSNSESEAESTENSPTPSPLQKKVTEDLSKTLLMYTVPAVQGFFRSISLSRGNNLQDTLRVLTLWFDYGHWPDVNEALVEGVKAIQIDTWLQVIPQLIARIDTPRPLVGRLIHQLLTDIGRYHPQALIYPLTVASKSTTTARHNAANKILKNMCEHSNTLVQQAMMVSEELIRVAILWHEMWHEGLEEASRLYFGERNVKGMFEVLEPLHAMMERGPQTLKETSFNQAYGRDLMEAQEWCRKYMKSGNVKDLTQAWDLYYHVFRRISKQLPQLTSLELQYVSPKLLMCRDLELAVPGTYDPNQPIIRIQSIAPSLQVITSKQRPRKLTLMGSNGHEFVFLLKGHEDLRQDERVMQLFGLVNTLLANDPTSLRKNLSIQRYAVIPLSTNSGLIGWVPHCDTLHALIRDYREKKKILLNIEHRIMLRMAPDYDHLTLMQKVEVFEHAVNNTAGDDLAKLLWLKSPSSEVWFDRRTNYTRSLAVMSMVGYILGLGDRHPSNLMLDRLSGKILHIDFGDCFEVAMTREKFPEKIPFRLTRMLTNAMEVTGLDGNYRITCHTVMEVLREHKDSVMAVLEAFVYDPLLNWRLMDTNTKGNKRSRTRTDSYSAGQSVEILDGVELGEPAHKKTGTTVPESIHSFIGDGLVKPEALNKKAIQIINRVRDKLTGRDFSHDDTLDVPTQVELLIKQATSHENLCQCYIGWCPFW

SEQ ID NO: 21 - DNA, 2344 bp, Homo sapiens
gacagctggaagagccgaacggataagagaagaggaggtgagaggagctgtacaccacaagaggcactgagggactcaggataacgggatgaagccgtcagtgcccccagaaacgaagcggccccggacgaatttctgagtcaccgtcgcgagaaagcgggctgagccgccattttgaagcctggcaaaccgaagcaagaaatgctgccgtgttggatctttgccagccttcgtgccgaatgggagcaggttggagggagggagagccaatatacactatgggctgattaagcccggttggctgccatgttgttaacgagcaccgatttcctctacttttgtcgaagaagtttattgtgggtcagggacgtcaggtcgcttgccttcgtttactgtggtcatgattgagcatatgaggacggccattattgttgggggcaaatggaaatgctctaggcggggccatttttcttaggggcaagctgtcgtcacccttgtcaactggttcggatgaagcccctgtggccgccatcttgatctcgggcggccccgataagggaggcggagtgtgcggagaggaggcggggcaactgcgcggacgtgacgcaaggcgccgccatgtcttttgagggcggtgacggcgccgggccggccatgctggctacgggcacggcgcggatggcgtcggggcgccccgaggagctgtgggaggccgtggtgggggccgctgagcgcttccgggcccggactggcacggagctggtgctgctgaccgcggccccgccgccaccaccccgcccgggcccctgtgcctatcctgcccatggtcgaggagccctggcggaggcagcgcgccgttgcctccacgacatcgcactggcccacagggctgccactgctgctcggcctcctgcgcccccaccagcaccacagccacccagtcccacacccagcccaccccggcctaccctggccagagaggacaacgaggaggacgaggatgagcccacagagacagagacctccggggagcagctgggcattagtgataatggagggctctttgtgatggatgaggacgccaccctccaggaccttccccccttctgtgagtcagaccccgagagtacagatgatggcagcctgagcgaggagacccccgccggcccccccacctgctcagtgcccccagcctcagccctacccacacagcagtacgccaagtccctgcctgtgtctgtgcccgtctggggcttcaaggagaagaggacagaggcgcggtcatcagatgaggagaatgggccgccctcttcgcccgacctggaccgcatcgcggcgagcatgcgcgcgctggtgctgcgagaggccgaggacacccaggtcttcggggacctgccacggccgcggcttaacaccagcgacttccagaagctgaagcggaaatattgaagtccagggagggagcgccccgggccgcgtccgccccgtcccacactacgcccccgccccactcccggggcctgctaatctgaggccgatccgggaccggcctccttgcgtctcccattcccaagattgtcccgcctctgccaatccccgccgtccttccagcccacgacctgccgcgccgaggagcggcatctgtcccgtttcccgattgggtctgtcgtctctctccgcctagcgacagattccttctattaagggattggctcgctgagttctaagctctaaatgggtcaactcctttgttttccgcctagcgacaagggatttgctcgcacggcattggctccatcccctagtcgctggacagctctttttttgattggctcaaatcctgtaaagggcttgaccagtctctacatagtcaccgtccgcttttcctgagttctccctcccaattggctccagcttcctgggggcgtggccaagccctcctcttcccagaattggcccggggccttcaatttacgttctttacactacggggactggggtcgtctttgcccacgtcccgacaacttgttccctgaccccctcagggatggccccaaactgtccctgcctctggcaccccctttcattggttccatccatccccacaacagcctgccaatcgaagcccgtccctgcatccaggatggtaccagctcccgcccctcgccccccacctccacaggtgccttaaagggccctcgtccacccaaggtggggggcaggggccctcactctccggccctggtgtgggggagagagtgaggggttgggggatcggcagttgggaggggcgctctgagattaaagagttttacctctgagataaaaaaaaaaaaaaaaaaaa

SEQ ID NO: 22 - protein, 256 aa, Homo sapiens
MASGRPEELWEAVVGAAERFRARTGTELVLLTAAPPPPPRPGPCAYAAHGRGALAEAARRCLHDIALAHRAATAARPPAPPPAPQPPSPTPSPPRPTLAREDNEEDEDEPTETETSGEQLGISDNGGLFVMDEDATLQDLPPFCESDPESTDDGSLSEETPAGPPTCSVPPASALPTQQYAKSLPVSVPVWGFKEKRTEARSSDEENGPPSSPDLDRIAASMRALVLREAEDTQVFGDLPRPRLNTSDFQKLKRKY

SEQ ID NO: 23 - DNA, 1964 bp, Homo sapiens
agacgcactgagcctaagcagccggtgatggcggcagcggctgtggtggctgcggcgggtccgggcccatgaggcgacgaaggaggcgggacggcttttacccagccccggacttccgagacagggaagctgaggacatggcaggagtgtttgacatagacctggaccagccagaggacgcgggctctgaggatgagctggaggaggggggtcagttaaatgaaagcatggaccatgggggagttggaccatatgaactgttttgcttttcttcccagtggcatggaacattgtgagaaatttgaaatctcagaaactagtgtgaacagagggccagaaaaaatcagaccagaatgttttgagctacttcgggtacttggtaaagggggctatggaaaggtttttcaagtacgaaaagtaacaggagcaaatactgggaaaatatttgccatgaaggtgcttaaaaaggcaatgatagtaagaaatgctaaagatacagctcatacaaaagcagaacggaatattctggaggaagtaaagcatcccttcatcgtggatttaatttatgcctttcagactggtggaaaactctacctcatccttgagtatctcagtggaggagaactatttatgcagttagaaagagagggaatatttatggaagacactgcctgcttttacttggcagaaatctccatggctttggggcatttacatcaaaaggggatcatctacagagacctgaagccggagaatatcatgcttaatcaccaaggtcatgtgaaactaacagactttggactatgcaaagaatctattcatggtggaacagtcacacacacattttgtggaacaatagaatacatggcccctgaaatcttgatgagaagtggccacaatcgtgctgtggattggtggagtttgggagcattaatgtatgacatgctgactggagcacccccattcactggggagaatagaaagaaaacaattgacaaaatcctcagatgtaaactcaatttgcctccctacctcacacaagaagccagagatctgcttaaaaagctgctgaaaagaaatgctgcttctcgtctgggagctggtcctggggacgctggagaagttcaagctcatccattctttagacacattaactgggaagaacttctggctcgaaaggtggagcccccctttaaacctctgttgcaatctgaagaggatgtaagtcagtttgattccaagtttacacgtcagacacctgtcgacagcccagatgactcaactctcagtgaaagtgccaatcaggtctttctgggttttacatatgtggctccatctgtacttgaaagtgtgaaagaaaagttttcctttgaaccaaaaatccgatcacctcgaagatttattggcagcccacgaacacctgtcagcccagtcaaattttctcctggggatttctggggaagaggtgcttcggccagcacagcaaatcctcagacacctgtggaatacccaatggaaacaagtggcatagagcagatggatgtgacaatgagtggggaagcatcggcaccacttccaatacgacagccgaactctgggccatacaaaaaacaagcttttcccatgatctccaaacggccagagcacctgcgtatgaatctatgacagagcaatgcttttaatgaatttaaggcaaaaaaggtggagagggagatgtgtgagcatcctgcaaggtgaaacgactcaaaatgacagtttcagagagtcaatgtcattacatagaacacttcagacacaggaaaaataaacgtggattttaaaaaatcaatcaatggtgcaaaaaaaaacttaaagcaaaatagtattgctgaactcttaggcacatcaattaattgattcctcgcgacatcttctcaaccttatcaaggattttcatgttgatgactcgaaactgacagtattaa

SEQ ID NO: 24 - protein, 525 aa, Homo sapiens
MRRRRRRDGFYPAPDFRDREAEDMAGVFDIDLDQPEDAGSEDELEEGGQLNESMDHGGVGPYELGMEHCEKFEISETSVNRGPEKIRPECFELLRVLGKGGYGKVFQVRKVTGANTGKIFAMKVLKKAMIVRNAKDTAHTKAERNILEEVKHPFIVDLIYAFQTGGKLYLILEYLSGGELFMQLEREGIFMEDTACFYLAEISMALGHLHQKGIIYRDLKPENIMLNHQGHVKLTDFGLCKESIHDGTVTHTFCGTIEYMAPEILMRSGHNRAVDWWSLGALMYDMLTGAPPFTGENRKKTIDKILKCKLNLPPYLTQEARDLLKKLLKRNAASRLGAGPGDAGEVQAHPFFRHINWEELLARKVEPPFKPLLQSEEDVSQFDSKFTRQTPVDSPDDSTLSESANQVFLGFTYVAPSVLESVKEKFSFEPKIRSPRRFIGSPRTPVSPVKFSPGDFWGRGASASTANPQTPVEYPMETSGIEQMDVTMSGEASAPLPIRQPNSGPYKKQAFPMISKRPEHLRMNL

SEQ ID NO: 25 - DNA, 6300 bp, Homo sapiens
cggccgcggtttccaggaggaagcaaggatgctttggacactgtgcgtggcgcctccgcggagcccccgcgctgccattcccggccgtcgctcggtcctccgctgacgggaagcaggaagtggcggcgggcgtcgcgagcggtgacatcacgggggcgacggcggcgaagggcgggggcggaggaggagcgagccgggccggggggcagctgcacagtctccgggatccccaggcctggaggggggtctgtgcgcggccggctggctctgccccgcgtccggtcccgagcgggcctccctcgggccagcccgatgtgaccgagcccagcggagcctgagcaaggagcgggtccgtcgcggagccggagggcgggaggaacatgacatcgcggagatggtttcacccaaatatcactggtgtggaggcagaaaacctactgttgacaagaggagttgatggcagttttttggcaaggcctagtaaaagtaaccctggagacttcacactttccgttagaagaaatggagctgtcacccacatcaagattcagaacactggtgattactatgacctgtatggaggggagaaatttgccactttggctgagttggtccagtattacatggaacatcacgggcaattaaaagagaagaatggagatgtcattgagcttaaatatcctctgaactgtgcagatcctacctctgaaaggtggtttcatggacatctctctgggaaagaagcagagaaattattaactgaaaaaggaaaacatggtagttttcttgtacgagagagccagagccaccctggagattttgttctttctgtgcgcactggtgatgacaaaggggagagcaatgacggcaagtctaaagtgacccatgttatgattcgctgtcaggaactgaaatacgacgttggtggaggagaacggtttgattctttgacagatcttgtggaacattataagaagaatcctatggtggaaacattgggtacagtactacaactcaagcagccccttaacacgactcgtataaatgctgctgaaatagaaagcagagttcgagaactaagcaaattagctgagaccacagataaagtcaaacaaggcttttgggaagaatttgagacactacaacaacaggagtgcaaacttctctacagccgaaaagagggtcaaaggcaagaaaacaaaaacaaaaatagatataaaaacatcctgccctttgatcataccagggttgtcctacacgatggtgatcccaatgagcctgtttcagattacatcaatgcaaatatcatcatgcctgaatttgaaaccaagtgcaacaattcaaagcccaaaaagagttacattgccacacaaggctgcctgcaaaacacggtgaatgacttttggcggatggtgttccaagaaaactcccgagtgattgtcatgacaacgaaagaagtggagagaggaaagagtaaatgtgtcaaatactggcctgatgagtatgctctaaaagaatatggcgtcatgcgtgttaggaacgtcaaagaaagcgccgctcatgactatacgctaagagaacttaaactttcaaaggttggacaagggaatacggagagaacggtctggcaataccactttcggacctggccggaccacggcgtgcccagcgaccctgggggcgtgctggacttcctggaggaggtgcaccataagcaggagagcatcatggatgcagggccggtcgtggtgcactgcagtgctggaattggccggacagggacgttcattgtgattgatattcttattgacatcatcagagagaaaggtgttgactgcgatattgacgttcccaaaaccatccagatggtgcggtctcagaggtcagggatggtccagacagaagcacagtaccgatttatctatatggcggtccagcattatattgaaacactacagcgcaggattgaagaagagcagaaaagcaagaggaaagggcacgaatatacaaatattaagtattctctagcggaccagacgagtggagatcagagccctctcccgccttgtactccaacgccaccctgtgcagaaatgagagaagacagtgctagagtctatgaaaacgtgggcctgatgcaacagcagaaaagtttcagatgagaaaacctgccaaaacttcagcacagaaatagatgtggactttcaccctctccctaaaaagatcaagaacagacgcaagaaagtttatgtgaagacagaatttggatttggaaggcttgcaatgtggttgactaccttttgataagcaaaatttgaaaccatttaaagaccactgtattttaactcaacaatacctgcttcccaattactcatttcctcagataagaagaaatcatctctacaatgtagacaacattatattttatagaatttgtttgaaattgaggaagcagttaaattgtgcgctgtattttgcagattatggggattcaaattctagtaataggcttttttatttttatttttatacccttaaccagtttaattttttttttcctcattgttggggatgatgagaagaaatgatttgggaaaattaagtaacaacgacctagaaaagtgagaacaatctcatttaccatcatgtatccagtagtggataattcattttgatggcttctatttttggccaaatgagaattaagccagtgcctgagactgtcagaagttgacctttgcactggcattaaagagtcatagaaaaagaatcatggatatttatgaattaaggtaagaggtgtggcttttttttttttcttttttccagccgttgaccaattatagttcggctgttgactgagaagtttgtggtgggaaaacgtttgccatattttctttgcatttgaataattgtcttgtacttagaaaaaaggcgtctatgaatgaccagtgtttttggtcgccaaatgttgctgacaaacttatcccaaaactttagtggcttaaaaaaacctgcccccaactgttagtcaatctgagctgggctcagctgggctgttcttctgccagcctgcaggtggccactcatgtggtcagcaggtcggcggagagactgggatggctgggcttctctctctgcctgcagtcctgagtctctccttcttcgtgtagtctctttcagtggcctggctggcagggtagctagacctctcacatgcagctcagagctcccaagagctcaaaagcagaaatggccaggccttctgaaaacttaagtccagaattgtcacagtgtcccttctacttccctctattgatgatgatgatgatgatgatgatgatgatgatgatgatgatgatggttttttctaatcagaagaaagctggggtatgccctctacttactaaacaagtcacaagcccagctcagattcaagaaaagggtgtgaagtagaggtgcagttaagtggggggccactagtctaacagacggtcacaaccagtgccatggaaaaccaaggatattagcaaaagcagaagttgctagtgaccttgggaagccgaagctgcttacagtagctgggacaagctgaaagtcagactaagaaataaagagagggccttcaagaagcttcctgaatgatttctgctagccctgagcctatttttggaaccagcacttggggaaactgatcttgtgaggatggatgtgtttagggacacagggcttttgagagcagcaccaccccactggggcatccccagacttgggaaacgtgactctttcttaatgccactgggttttagtcaggccacagtgagaaggaacagccctaacaggcctccagccaggttgaatgagctcatttttgttgtagccaaccagtaagatttgctaatgttctacattaagtgccttctccaaagacatccctctttgcctcatatgttgaatcatccagtgcggatatttcaatgaaaatatcattggttgacttttgtgatggtaataatgctatggcatctttgccatgaagttgtggcctccttggattcttctgactttggcttctgaaaggaaggcctagatccagccctggtggtagttcctttctgaggtctctcagtcccttgagactttggggtagtttggctgccattctcactgacaaaatgtatatcagcccccacctccaccccccaatattccttgaactttgaattgcttcagaacacaggtgtggcctgaaggtattcccttattagggaagtgtcactgctgtcttctagtcaaacttgtaaagaaaaagattccagttcagtatttgcagcaagaagcttgaatgctgttctttttatcgcattgttacatcgactcattctccattttgctttggttttgtcttgacttgacttgactttgggggtaaagtctttcaccagcacacaagagtttgattgtacaaatatatcttctgcattaacatctctgcctgttgcttaagatcagttgcttttatactcagaatggaaatacctgatcttggctagttttgttataagatattgatttcatttagatttccctccacgaggtcagcaaactatcatgttcttatgtaaacttaggccaaggccagagttatcatagtccctaggttgctacggcttatcatgtgcttggtaaaaggtgatcgcaggttctcagacgagtttactttacatgagatggaatcaggcagagaggctgggatgatggagaaagctcgaggtgaagttttaaaaaaaaagttgtggaaaggaaagttccaaagaggtggtttctgaggaagtcagagcgcccagggccagagcagtcagtaatgggtgaatgaggttgtttggaaagtcggtgtgacagacacatggatgccatctacttctaggttgctggtgggtattaaatatgcacaatattccatagctcactgaggattttaaaattataagcataggattttatattttggggtgaaagaattatctggcacattaggtattggagtttaaaaaaaaagccaaatttcacagtcttaataactttttttaaaaaaaactaaaaggcgcttcatgtccagtgtgtggcccttctgaaacttatggtcatctctcccactgaaaccaaggtcttttcaaatgtggctaaatggggatgaggagacacgggtaggactttcttggtgtgtgtgcattctttaaagagccaagttgcttcggggaaacagccaggaaaatggtcaagattatttttagaggttattttattggggattttaagaactaataacatcttgagttatttttaattcagggggatgtggaaaggtttgcaattgtcaagtgttttgttgtagcttagtatccataagggaaacttagactatagacataactacaaagccagtgcagcttttgttttctgtatgttgttgggggatcaactttcacacatagcaagcacatggcctccctgatgtcaggatgcctttgttaggatctgtatttgcccttaattttgttgaaatcttttttccttcttcctcttgaaaagttccaaaatatagtttattgtatctttcatcactaaaaatttgttcctttttcactatgggcagttcacacaaggcaaaaactattgaacagttggttttagtgtgttgtataactttgctgtatatcaaactaattttgacaagttttcatcctaagcctcaaatcatgtaattaataatttgcctgtttatttatgacctaattgtgattcttttattaataaaagctaatgggaaaaggatccctgattaagctgatgactagacctacaattaattttcctgcagtatatgaagtattgtaccagagtattaaaagatatgtaatattttattgataaatctatcctttaaaaggaatacgttttaggatgtcatcattttgatgtgaatcatgtaaatgttgataatatgctgtttattatacatttagtgtttcaagagattcacttaattgcctttttgcccacgtatattatgtagtctatttgcaactgttcttaaaaaaatgacattaaaagaatagtttatgtagagaaacattagtggatgttaattgtctccccacctatatttatgggtgttagcgcaactgctttgctagttgcaaagctgtattatcagagtaaaagtgtatttgtaaactgtatgggaactaaaaattaggaataaaaccattttcttatatgaaaaaaaaaaaaaaaaaa

SEQ ID NO: 26 - DNA, 2069 bp, Homo sapiens
cggccgcggtttccaggaggaagcaaggatgctttggacactgtgcgtggcgcctccgcggagcccccgcgctgccattcccggccgtcgctcggtcctccgctgacgggaagcaggaagtggcggcgggcgtcgcgagcggtgacatcacgggggcgacggcggcgaagggcgggggcggaggaggagcgagccgggccggggggcagctgcacagtctccgggatccccaggcctggaggggggtctgtgcgcggccggctggctctgccccgcgtccggtcccgagcgggcctccctcgggccagcccgatgtgaccgagcccagcggagcctgagcaaggagcgggtccgtcgcggagccggagggcgggaggaacatgacatcgcggagatggtttcacccaaatatcactggtgtggaggcagaaaacctactgttgacaagaggagttgatggcagttttttggcaaggcctagtaaaagtaaccctggagacttcacactttccgttagaagaaatggagctgtcacccacatcaagattcagaacactggtgattactatgacctgtatggaggggagaaatttgccactttggctgagttggtccagtattacatggaacatcacgggcaattaaaagagaagaatggagatgtcattgagcttaaatatcctctgaactgtgcagatcctacctctgaaaggtggtttcatggacatctctctgggaaagaagcagagaaattattaactgaaaaaggaaaacatggtagttttcttgtacgagagagccagagccaccctggagattttgttctttctgtgcgcactggtgatgacaaaggggagagcaatgacggcaagtctaaagtgacccatgttatgattcgctgtcaggaactgaaatacgacgttggtggaggagaacggtttgattctttgacagatcttgtggaacattataagaagaatcctatggtggaaacattgggtacagtactacaactcaagcagccccttaacacgactcgtataaatgctgctgaaatagaaagcagagttcgagaactaagcaaattagctgagaccacagataaagtcaaacaaggcttttgggaagaatttgagacactacaacaacaggagtgcaaacttctctacagccgaaaagagggtcaaaggcaagaaaacaaaaacaaaaatagatataaaaacatcctgccctttgatcataccagggttgtcctacacgatggtgatcccaatgagcctgtttcagattacatcaatgcaaatatcatcatgcctgaatttgaaaccaagtgcaacaattcaaagcccaaaaagagttacattgccacacaaggctgcctgcaaaacacggtgaatgacttttggcggatggtgttccaagaaaactcccgagtgattgtcatgacaacgaaagaagtggagagaggaaagagtaaatgtgtcaaatactggcctgatgagtatgctctaaaagaatatggcgtcatgcgtgttaggaacgtcaaagaaagcgccgctcatgactatacgctaagagaacttaaactttcaaaggttggacaagggaatacggagagaacggtctggcaataccactttcggacctggccggaccacggcgtgcccagcgaccctgggggcgtgctggacttcctggaggaggtgcaccataagcaggagagcatcatggatgcagggccggtcgtggtgcactgcaggtgacagctcctgctgcccctctaggccacagcctgtccctgtctcctagcgcccagggcttgcttttacctacccactcctagctctttaactgtaggaagaatttaatatctgtttgaggcatagagcaactgcattgagggacattttgatcccaaggcatatttctcctagaccctacagcactgccattggccatggccatggcaacatgctcagttaaaacagcaaagactaagtcagcattatctctgagtccaccagaagttgtgcattaaacaacttcatcctggaaaaaaaaaaaaaaaa

SEQ ID NO: 27 - protein, 593 aa, Homo sapiens [sequence omitted]

SEQ ID NO: 28 - protein, 460 aa, Homo sapiens
MTSRRWFHPNITGVEAENLLLTRGVDGSFLARPSKSNPGDFTLSVRRNGAVTHIKIQNTGDYYDLYGGEKFATLAELVQYYMEHHGQLKEKNGDVIELKYPLNCADPTSERWFHGHLSGKEAEKLLTEKGKHGSFLVRESQSHPGDFVLSVRTGDDKGESNDGKSKVTHVMIRCQELKYDVGGGERFDSLTDLVEHYKKNPMVETLGTVLQLKQPLNTTRINAAEIESRVRELSKLAETTDKVKQGFWEEFETLQQQECKLLYSRKEGQRQENKNKNRYKNILPFDHTRVVLHDGDPNEPVSDYINANIIMPEFETKCNNSKPKKSYIATQGCLQNTVNDFWRMVFQENSRVIVMTTKEVERGKSKCVKYWPDEYALKEYGVMRVRNVKESAAHDYTLRELKLSKVGQGNTERTVWQYHFRTWPDHGVPSDPGGVLDFLEEVHHKQESIMDAGPVVVHCR